(19) United States
(12) Patent Application Publication
(10) Pub. No.: US 2019/0022212 A1
(43) Pub. Date: Jan. 24, 2019

(54) METHODS FOR SAFE INDUCTION OF CROSS-CLADE IMMUNITY AGAINST HUMAN IMMUNODEFICIENCY VIRUS INFECTION IN HUMAN

(71) Applicants: Beth Israel Deaconess Medical Center, Inc., Boston, MA (US); Janssen Vaccines & Prevention B.V., Leiden (NL)

(72) Inventors: Dan BAROUCH, Newton, MA (US); Johanna SCHUITEMAKER, Amstelveen (NL); Maria Grazia PAU, Leiden (NL); Danielle VAN MANEN, Leiden (NL); Frank TOMAKA, Titusville, NJ (US); Jennifer Anne HENDRIKS, Poeldijk (NL)

(21) Appl. No.: 16/041,167

(22) Filed: Jul. 20, 2018

Related U.S. Application Data
(60) Provisional application No. 62/535,458, filed on Jul. 21, 2017.

(51) Int. Cl. A61K 39/21; A61K 39/295; C07K 14/16

(52) U.S. Cl. CPC A61K 39/21; A61K 2039/545; C07K 14/16; A61K 39/295

(57) ABSTRACT

Methods for inducing safe and effective immune response against multiple clades of Human Immunodeficiency Virus (HIV) infection in human subjects are described. The methods involve heterologous vaccine combinations of adenovirus serotype 26 expression vectors expressing at least three mosaic HIV antigens with at least one isolated HIV gp140 protein.

### STATEMENT REGARDING FEDERALLY SPONSORED RESEARCH OR DEVELOPMENT

This invention was made with government support under Grant Nos. AI078526 and AI096040 awarded by the National Institutes of Health. The government has certain rights in the invention.

### REFERENCE TO SEQUENCE LISTING SUBMITTED ELECTRONICALLY

This application contains a sequence listing, which is submitted electronically via EFS-Web as an ASCII formatted sequence listing with a file name “688097_354US Sequence Listing”, creation date of Jul. 19, 2017, and having a size of about 54 kB. The sequence listing submitted via EFS-Web is part of the specification and is herein incorporated by reference in its entirety.

### FIELD OF THE INVENTION

This invention relates to methods for safely inducing effective immunity against a wide variety of human immunodeficiency virus (HIV) subtypes responsible for HIV infections globally in human subjects. In particular, the invention relates to heterologous vaccine combinations of adenovirus serotype 26 expression vectors expressing at least three mosaic HIV antigens with at least one isolated HIV gp140 protein to provide safe induction of effective immunity against multiple clades of HIV in human subjects.

### BACKGROUND OF THE INVENTION

Human Immunodeficiency Virus (HIV) affects millions of people worldwide, and the prevention of HIV remains a very high priority, even in an era of widespread antiretroviral treatment. It is an urgent global health priority to find a safe and potent HIV vaccine that would prevent HIV infection or blunt its initial impact prior to diagnosis, including both destruction of the gut CD4 pool [3] and high risk of transmission [4].

The ability of HIV to evade host immunity and constantly mutate makes development of a preventive vaccine enormously challenging [84]. A fully efficacious vaccine is anticipated to be able to elicit both potent cellular responses and broadly neutralizing antibodies capable of neutralizing HIV-1 variants from different clades.

Broadly neutralizing antibodies are directed against highly conserved regions in the viral envelope. Until recently, most anti-HIV vaccines used purified HIV antigenic proteins, such as gp160, gp41 or gp120 presented in a soluble form. Most envelope (Env) protein-based immunogens are monomeric envelope molecules that elicit binding antibodies, but not potent neutralizing antibodies. This is in part due to the fact that neutralizing antibodies recognize tertiary and quaternary epitopes on the native, trimeric structure of the viral envelope proteins. In addition, most monomeric Env-based immunogens do not induce a cell-mediated response. It was reported that stabilized trimers of HIV-1 Env induced broadly neutralizing antisera against HIV-1 in vivo. See, e.g., US 2012/0045472.

In order to elicit both potent cellular responses and broadly neutralizing antibodies, recombinant vectors have been used to express genes for HIV antigenic proteins in vivo as an alternative to live attenuated viral vaccines. The use of replication incompetent recombinant viral vectors has been explored for vaccines and gene therapy. In particular, replication incompetent recombinant adenoviral vectors, particularly adenovirus serotype 5 (Ad5), have been extensively studied for gene delivery applications, including vaccination. Although such replication incompetent Ad5 vector-based vaccines have been shown to elicit protective immune responses in a variety of animal models, the utility of recombinant Ad5 vector-based vaccines for HIV and other pathogens can be limited by the high seroprevalence of Ad5-specific neutralizing antibodies (NAbs) in human populations [17]. For example, in a seroepidemiology study of 4,381 subjects worldwide, it was observed that Ad5 NAb titers were nearly universal and high titer in sub-Saharan Africa, with the majority of individuals exhibiting Ad5 NAb titers >200 [14].

Several HIV-1 vaccine efficacy trials have been conducted using vaccines based on recombinant Ad5 vector-based vaccines. These studies include the HVTN 502/STEP (Merck Ad5), HVTN 503/Phambili (Merck Ad5), and HVTN 505 (NIH VRC DNA/Ad5) HIV-1 vaccine efficacy trials. However, all three of these HIV-1 vaccine efficacy studies, which utilized nonreplicating Ad5 and DNA/Ad5 vaccines, showed no efficacy against HIV-1 infection. Moreover, a trend towards increased HIV-1 infection was observed in subjects vaccinated with the Merck Ad5 vaccine from the STEP study as compared with placebo. Experience to date with replication incompetent vectors such as adenovirus subtype 5 for HIV vaccine has been disappointing, with failure to show benefit in several efficacy trials [5-8].

Concerns regarding the safety of Ad5 vectors, particularly from the STEP study [8, 10], have led to the exploration of biologically substantially different Ad vectors from alternative serotypes as viral vaccine vectors [11-13]. One example of an alternative adenovirus serotype to Ad5 is Adenovirus serotype 26 (Ad26). Ad26 is a relatively uncommon virus in humans, and is not known to replicate in any other species. A number of surveys for adenovirus in different populations have shown it to be isolated only rarely, and even when isolated, seldom associated with symptoms. Experimental immunization, likewise, showed little evidence for serious infection. See, e.g., references [14], and [27]-[43]. Thus, there is no evidence from observational studies that Ad26 causes clinical symptoms in healthy adults, and experimental data from an Ad26 challenge study also suggested that enteric Ad26 infection does not produce symptoms [44]. Replication-defective adenovirus vectors, rAd26, can be grown to high titers in Ad5 E1-complementing cell lines suitable for manufacturing these vectors at a large scale and at clinical grade [11], and this vector has been shown to induce humoral and cell-mediated immune responses in prime-boost vaccine strategies [11, 21].

These alternative adenovirus vectors show efficient transduction of human dendritic cells [63, 22], and thus have the capability to mediate high level antigen delivery and presentation.

In terms of at least receptor usage, in vivo tropism, interactions with dendritic cells, innate immune profiles, adaptive immune phenotypes, and protective efficacy against SIV in rhesus monkeys, Ad26 has proven to be biologically very different from Ad5 [11, 12, 15, 19-22]. Moreover, the safety and immunogenicity of nonreplicating Ad26 vector in humans have been demonstrated (ClinicalTrials.Gov NCT01215149).

Modified Vaccinia Ankara (MVA) virus, a replication-deficient strain of vaccinia virus, has also been used as a viral vector for recombinant expression of HIV antigenic proteins. See, e.g., US20110159036, U.S. Pat. No. 8,197,825, etc. MVA is related to Vaccinia virus, a member of the genera Orthopoxvirus in the family of Poxviridae. Poxviruses are known to be good inducers of CD8 T cell responses because of their intracytoplasmic expression. However, they are generally believed to be poor at generating CD4 MHC class II restricted T cells. See, e.g., [64].

One possible drawback of replication-incompetent viral vectors is that expression of the target gene to be delivered to the host from the viral vector can decrease following administration of the vector. Being unable to replicate or propagate in the host, the viral vector cannot produce any new copies that can subsequently be used to augment gene expression, thus requiring re-administration of the viral vector. If the same adenovirus serotype is re-administered to the host, the host can generate neutralizing antibodies to that particular adenovirus serotype, resulting in a serotype specific anti-adenovirus response. Such a serotype specific anti-adenovirus response can prevent effective re-administration of the viral vector, rendering it less effective, maybe also less safe, as a vaccine or gene delivery vehicle.

Some potential HIV vaccine regimens using viral vectors and protein were disclosed in WO 2016/049287.

Partial efficacy in preventing HIV acquisition in humans was reported from a recent clinical study with a regimen consisted of a recombinant canarypox vector vaccine prime (ALVAC-HIV, Sanofi Pasteur) and a gp120 protein boost (AIDSVAX B/E, Global Solutions for Infectious Diseases) [9, 84]. However, no significant difference in the HIV-1 viral load or the postinfection CD4+ count was observed from the study [9]. This study indicates that non-neutralizing Env-specific antibodies may have some protective capacity that is linked to subtype-specific antibody function [9].

Vaccine or therapeutic strategies aimed at preventing HIV infection must act aggressively to clear the very first infected cells quickly due to the fact that once inside a cell, HIV has evolved intricate means to hide from the immune system. It has been suggested that next generation vaccine efforts should place some emphasis on generation antibodies that not only enhance antibody-dependent cellular cytotoxicity (ADCC), but also antibody-dependent cellular phagocytosis (ADCP) [Ackerman et al., Curr HIV Res. (2013) 11(5): 365-377]. It was suggested that specific features, such as IgG subclass and/or glycosylation state, rather than prevalence of HIV-specific antibodies, may account for the enhanced phagocytic activity [Ackerman et al., J Virol. (2013) 87(10): 5468-5476]. It was shown that antibody glycosylation is determined in an antigen- and pathogen-specific manner during HIV infection, and that distinct vaccine regimens induced different antigen-specific IgG glycosylation profiles [Mahan et al., PLoS Pathog. 2016 Mar. 16; 12(3):e1005456].

Accordingly, there is a need in the art for improved methods for generating immune responses that are safe, long-acting, and effective against a wide diversity of circulating types of HIV transmission, including the most frequent for multiple regions of the world, in human subjects.

### BRIEF SUMMARY OF THE INVENTION

The invention is based in part on the discovery that combinations of an isolated HIV antigenic protein with expression vectors, such as replication incompetent viral vectors, encoding HIV antigens, induce safe and effective immune response against multiple clades of HIV infection in healthy human subjects.

Accordingly, one general aspect of the invention relates to a method of inducing a safe and effective immune response against multiple clades of human immunodeficiency virus (HIV) in a human subject in need thereof, comprising:

(1) administering to the subject a priming composition comprising one or more Ad26 vectors together encoding at least three HIV antigenic polypeptides having the amino acid sequences of SEQ ID NO: 1, SEQ ID: NO 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors;

(2) administering to the subject, about 10-14 weeks after (1), the priming composition at a total dose of about 5×10⁹ to about 1×10¹¹ vp, preferably about 5×10¹⁰ vp, of the Ad26 vectors;

(3) administering to the subject, about 22-26 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 125 μg to 350 μg, preferably about 250 μg, of the at least one isolated HIV envelope glycoprotein;

(4) administering to the subject, together with (3), a second boosting composition comprising one or more Ad26 vectors together encoding the at least three HIV antigenic polypeptides and a pharmaceutically acceptable carrier, at a total dose of about 5×10⁹ to about 1×10¹¹ vp, preferably about 5×10¹⁰ vp, of the Ad26 vectors; or administering to the subject, together with (3), a second alternative boosting composition comprising one or more MVA vectors together encoding the at least three HIV antigenic polypeptides, and a pharmaceutically acceptable carrier, at a total dose of about 10⁷ to about 10⁹ plaque-forming units (pfu), preferably about 10⁸ pfu, of the MVA vectors,

wherein the safe and effective immune response comprises at least one selected from the group consisting of:
a. an ADCP response to isolated HIV envelope glycoproteins of clades B and C at a median response rate of at least 56%; preferably a median response rate of about 72%; more preferably a median response rate of about 80%; even more preferably further an ADCP response to isolated HIV envelope glycoproteins of clade A at a median response rate of at least 35%;
b. a humoral immune response against HIV envelope glycoproteins from clades A, B, and C at a median response rate of at least 90%, preferably at a median response rate of about 100%, as measured by IgG bindings to HIV envelope glycoproteins from clades A, B and C in enzyme-linked immunosorbent assays (ELISAs); and
c. a cellular immune response at a median response rate of at least 50% as measured by a γIFN response in an enzyme-linked immunospot assay (ELISPOT) to a potential T-cell epitopes (PTE) peptide pool.

In certain preferred embodiments, the method further comprises:

(5) repeating steps (3) and (4), at about 42-60 weeks, e.g. at about 48 weeks, after step (1).

In preferred embodiments, the composition at step (4), and step (5) if present, is the second boosting composition that comprises the one or more Ad26 vectors (i.e. is not the second alternative boosting composition that comprises the one or more MVA vectors).

In certain embodiments, a fourth HIV antigenic polypeptide having the amino acid sequence of SEQ ID NO: 8 is encoded by the one or more Ad26 vectors in the priming composition and/or in the second boosting composition, and/or by the one or more MVA vectors in the second alternative boosting composition.

In certain embodiments, the first boosting composition further comprises an isolated HIV envelope glycoprotein having the amino acid sequence of SEQ ID NO: 6.

In a preferred embodiment, the invention relates to a method of inducing a safe and effective immune response against multiple clades of human immunodeficiency virus (HIV) in a human subject uninfected by HIV, comprising:

(1) administering to the subject a priming composition comprising at least three Ad26 vectors together encoding at least three HIV antigenic polypeptides having the amino acid sequences of SEQ ID NO: 1, SEQ ID: NO 3, and SEQ ID NO: 4, respectively, and a pharmaceutically acceptable carrier, in a total dose of about 5×10¹⁰ viral particles (vp) of the Ad26 vectors;

(2) administering to the subject, about 12 weeks after (1), the priming composition at a total dose of about 5×10¹⁰ vp of the Ad26 vectors;

(3) administering to the subject, about 24 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 250 μg of the at least one isolated HIV envelope glycoprotein;

(4) administering to the subject, together with (3), a second boosting composition comprising the at least three Ad26 vectors and a pharmaceutically acceptable carrier, at a total dose of about 5×10¹⁰ vp of the Ad26 vectors;

(5) administering to the subject, about 48 weeks after (1), the first boosting composition at a total dose of about 250 μg of the at least one isolated HIV envelope glycoprotein; and

(6) administering to the subject, together with (5), the second boosting composition at a total dose of about 5×10¹⁰ vp of the Ad26 vectors;

wherein the safe and effective immune response comprises at least one selected from the group consisting of:
a. an ADCP response to isolated HIV envelope glycoproteins of clades B and C at a median response rate of at least 56%; preferably a median response rate of about 72%; more preferably a median response rate of about 80%; even more preferably further an ADCP response to isolated HIV envelope glycoproteins of clade A at a median response rate of at least 35%;
b. a humoral immune response against HIV envelope glycoproteins from clades A, B, and C at a median response rate of at least 90%, preferably at a median response rate of about 100%, as measured by IgG bindings to HIV envelope glycoproteins from clades A, B and C in enzyme-linked immunosorbent assays (ELISAs); and
c. a cellular immune response at a median response rate of at least 50% as measured by a γIFN response in an enzyme-linked immunospot assay (ELISPOT) to a potential T-cell epitopes (PTE) peptide pool, preferably at a median response rate of about 77%, more preferably at a median response rate of about 83%.

According to an embodiment of the invention, a method of the invention induces at least two of the responses in a to c above. Preferably, a method of the invention induces the responses in a, b and c.

According to embodiments of the invention, a method of the invention induces an ADCP response to isolated HIV envelope glycoproteins of clades B and C in human subjects at a median response rate of at least about 56%, 58%, 60%, 62%, 64%, 66%, 68%, 70%, 72%, 74%, 76%, 78%, 80% or more. In a preferred embodiment, the method also induces an ADCP response to an isolated HIV envelope glycoprotein of clade A in human subjects at a median response rate of at least about 35%, 40%, or more.

According to embodiments of the invention, a method of the invention induces a humoral immune response against HIV envelope glycoprotein from clades A, B, and C at a median response rate of at least about 90%, 92%, 94%, 96%, 98% or 100%, preferably at a median response rate of about 100%.

According to embodiments of the invention, a method of the invention induces a cellular immune response as measured by a γIFN response in an ELISPOT at a median response rate of at least about 50%, 55%, 60%, 65%, 70%, 75%, 80%, 85% or more.

According to embodiments of the invention, a method of the invention induces a safe and effective immune response that comprises a persistent humoral immune response against HIV envelope glycoprotein from at least Clade C at a response rate of at least 90%, 95% or 100% at 48 weeks after the last administration of the boosting compositions. According to certain embodiments, a method of the invention induces a safe and effective immune response that comprises a persistent cellular immune response against HIV Env, as can be measured by γIFN ELISPOT responses to PTE Env peptides, at 48 weeks after the last administration of the boosting compositions.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIG. 1 shows the results from a SHIV challenge experiment with the vaccinated NHPs;

FIG. 2 shows that binding antibodies to HIV Env together with HIV Env specific T cells correlate with protection in NHP SHIV_{SF162P3} challenge study (Shaded colors and diagonal lines indicate the probability of infection modeled on ELISpot and ELISA responses);

FIG. 3 shows the results of total IgG gp140 ENV ELISA Clade C (C97ZA.012) from a human clinical study (Week 52 Analysis); in the top of each segment the components of the boost are indicated: gp140 LD and HD mean 50 μg and 250 μg doses, respectively;

FIG. 4 shows the results of total IgG gp140 ENV ELISA Clade A (92UG037.1) from the human clinical study (Week 52 Analysis); in the top of each segment the components of the boost are indicated: gp140 LD and HD mean 50 μg and 250 μg doses, respectively;

FIG. 5 shows the results of total IgG gp140 ENV ELISA Clade B (1990a) from the human clinical study (Week 52 Analysis); in the top of each segment the components of the boost are indicated: gp140 LD and HD mean 50 μg and 250 μg doses, respectively;

FIG. 6 shows the results of total IgG gp140 ENV ELISA Clade C (Consensus) from the human clinical study (Week 52 Analysis); in the top of each segment the components of the boost are indicated: gp140 LD and HD mean 50 μg and 250 μg doses, respectively;

FIG. 7 shows the results of total IgG gp140 ENV ELISA Clade C (Mosaic construct, Mos1) from the human clinical study (Week 52 Analysis); in the top of each segment the components of the boost are indicated: gp140 LD and HD mean 50 μg and 250 μg doses, respectively;

FIG. 8 shows the results of total IgG gp140 ENV ELISA Clade C (C97ZA.012) over time in group treated with the regimen of Ad26/AD26+gp140 HD in the human clinical study (Week 52 Analysis);

FIG. 9 shows the results of HIV-1 tier 1 TZM-bl neutralization assays against Clade C (MW965.26) from the human clinical study (Week 52 Analysis); in the top of each segment the components of the boost are indicated: gp140 LD and HD mean 50 μg and 250 μg doses, respectively;

FIG. 10 shows the results of ADCP Env gp140 Clade C (C97ZA.012) from the human clinical study (Week 52 Analysis); in the top of each segment the components of the boost are indicated: gp140 LD and HD mean 50 μg and 250 μg doses, respectively;

FIG. 11 shows the results of IFNg ELISPOT ENV PTE peptide pool (from the human clinical study (Week 52 Analysis); in the top of each segment the components of the boost are indicated: gp140 LD and HD mean 50 μg and 250 μg doses, respectively;

FIG. 12 shows the results of ICS (FHCRC) on CD4⁺ T-cells expressing IFNg and/or IL2 to HIV ENV gp120 peptide pool 1 (Mos 1); in the top of each segment the components of the boost are indicated: gp140 LD and HD mean 50 μg and 250 μg doses, respectively;

FIG. 13 shows the results of total IgG gp140 ENV ELISA Clade C (C97ZA.012) from a human clinical study (Week 96 Analysis); in the figure legend the components of the boost are indicated: gp140 LD and HD mean 50 μg and 250 μg doses, respectively; and

FIG. 14 shows the results of total IgG gp140 ENV ELISA Clade C (Mosaic construct, Mos1) from the human clinical study (Week 96 Analysis); in the figure legend the components of the boost are indicated: gp140 LD and HD mean 50 μg and 250 μg doses, respectively.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means any animal, preferably a mammal, most preferably a human, whom will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

An “adenovirus capsid protein” refers to a protein on the capsid of an adenovirus (e.g., Ad26 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton and fiber are of Ad26.

As used herein, the term “co-delivery” or “administered together with” refers to simultaneous administration of two components, such as a viral expression vector and an isolated antigenic polypeptide. “Simultaneous administration” can be administration of the two components at least within the same day. When two components are “administered together with,” they can be administered in separate compositions sequentially within a short time period, such as 24, 20, 16, 12, 8 or 4 hours, or within 1 hour, or they can be administered in a single composition at the same time.

The terms “adjuvant” and “immune stimulant” are used interchangeably herein, and are defined as one or more substances that cause stimulation of the immune system. In this context, an adjuvant is used to enhance an immune response to the antigenic polypeptides of the invention, being the antigenic HIV antigenic polypeptides of the invention.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., human immunodeficiency virus (HIV) and certain species of adenovirus, prions, bacteria, fungi, protozoa and the like.

As used herein, “a method of inducing safe and effective immune response” or “a safe method of inducing an effective immune response” means a method to induce an immune response that is effective to provide benefits of a vaccine, without causing unacceptable vaccine related adverse events, when administered to the human subject.

As used herein, the phrase “unacceptable vaccine related adverse events,” “unacceptable adverse events,” and “unacceptable adverse reaction,” shall all mean harm or undesired outcome associated with or caused by the administration of a vaccine, and the harm or undesired outcome reaches such a severity that a regulatory agency deems the vaccine unacceptable for the proposed use.

As used herein, an “effective immune response” refers to an immune response that is required or contribute to the prevention or treatment of HIV infection in a human subject. Examples of effective immune responses include, but are not limited to, a humoral immunogenicity against HIV, such as an ADCP response to an isolated HIV envelope glycoprotein, IgG binding to HIV envelope glycoproteins as measured by ELISA, a cellular immune response as measured by a γIFN response in an ELISPOT to a potential T-cell epitopes (PTE) peptide pool, etc. An “effective immune response” can but does not necessarily refer to protective immunity in a human subject.

As used herein, a “response rate” refers to the number of subjects who have responded to a treatment with a particular outcome divided by the number of treated subjects.

As used herein, a “potential T-cell epitopes (PTE) peptide pool” refers to a pool of peptides containing potential T-cell epitope (PTE) peptides embedded in antigenic protein sequences of circulating strains of HIV-1 worldwide. Examples of a “potential T-cell epitopes (PTE) peptide pool” include, but are not limited to, a HIV-1 PTE Gag peptide pool, a HIV-1 PTE Env peptide pool, and a HIV-1 PTE Pol peptide pool, which are available from the U.S. National Institute of Health AIDs Reagent Program.

Human immunodeficiency virus (HIV) is a member of the genus Lentivirinae, which is part of the family of Retroviridae. Two species of HIV infect humans: HIV-1 and HIV-2. HIV-1 is the most common strain of HIV virus, and is known to be more pathogenic than HIV-2. As used herein, the terms “human immunodeficiency virus” and “HIV” refer, but are not limited to, HIV-1 and HIV-2, preferably HIV-1.

HIV is categorized into multiple clades with a high degree of genetic divergence. As used herein, the term “HIV clade” or “HIV subtype” refers to related human immunodeficiency viruses classified according to their degree of genetic similarity. There are currently three groups of HIV-1 isolates: M, N and O. Group M (major strains) consists of at least ten clades, A through J. Group O (outer strains) can consist of a similar number of clades. Group N is a new HIV-1 isolate that has not been categorized in either group M or O. In certain exemplary embodiments, a broadly neutralizing antibody described herein will recognize and raise an immune response against two, three, four, five, six, seven, eight, nine, ten or more clades and/or two or more groups of HIV.

It is discovered in the invention that heterologous prime-boost combinations, in particular, priming with an expression vector, such as rAd26, encoding one or more HIV antigenic proteins, followed by boosting with an isolated HIV antigenic protein, such as an HIV envelope glycoprotein, in combination with rAd26 or MVA encoding one or more HIV antigenic proteins, are surprisingly effective in generating protective immune responses against one or more subtypes of HIV in non-human primates, and in generating effective immune responses against at least clades A, B and C of HIV in humans.

HIV Antigenic Proteins

As used herein, the term “antigenic polypeptide of an HIV,” “HIV antigenic polypeptide,” “HIV antigenic protein,” “HIV immunogenic polypeptide,” or “HIV immunogen” refers to a polypeptide capable of inducing an immune response, e.g., a humoral and/or cellular mediated response, against the HIV in a subject in need thereof. The antigenic polypeptide can be a protein of the HIV, a fragment or epitope thereof, or a combination of multiple HIV proteins or portions thereof, that can induce an immune response or produce an immunity, e.g., protective immunity, against the HIV in a subject in need thereof.

Preferably, an antigenic polypeptide is capable of raising in a host a protective immune response, e.g., inducing an immune response against a viral disease or infection, and/or producing an immunity in (i.e., vaccinates) a subject against a viral disease or infection, that protects the subject against the viral disease or infection. For example, the antigenic polypeptide can comprise a protein or fragments thereof from HIV, such as the HIV envelope gp160 protein, the HIV matrix/capsid proteins, and the HIV gag, pol and env gene products.

According to embodiments of the invention, the antigenic polypeptide can be an HIV-1 antigen or fragments thereof. Examples of HIV antigens include, but are not limited to gag, pol, and env gene products, which encode structural proteins and essential enzymes. Gag, pol, and env gene products are synthesized as polyproteins, which are further processed into multiple other protein products. The primary protein product of the gag gene is the viral structural protein gag polyprotein, which is further processed into MA, CA, SP1, NC, SP2, and P6 protein products. The pol gene encodes viral enzymes (Pol, polymerase), and the primary protein product is further processed into RT, RNase H, IN, and PR protein products. The env gene encodes structural proteins, specifically glycoproteins of the virion envelope. The primary protein product of the env gene is gp160, which is further processed into gp120 and gp41.

According to a preferred embodiment, the antigenic polypeptide comprises an HIV Gag, Env, or Pol antigen, or any portion or combination thereof, more preferably an HIV-1 Gag, Env, or Pol antigen or any portion or combination thereof.

According to another preferred embodiment, the antigenic polypeptide or a peptide encoded by a vector according to the invention is a mosaic HIV antigen. As used herein, “mosaic antigen” refers to a recombinant protein assembled from fragments of natural sequences. The “mosaic antigen” can be computationally generated and optimized using a genetic algorithm. Mosaic antigens resemble natural antigens, but are optimized to maximize the coverage of potential T-cell epitopes found in the natural sequences, which improves the breadth and coverage of the immune response.

A mosaic HIV antigen according to the invention is preferably a mosaic Gag-Pol-Env antigen, and more preferably a mosaic HIV-1 Gag-Pol-Env antigen. As used herein, “a mosaic HIV Gag-Pol-Env antigen” specifically refers to a mosaic antigen comprising multiple epitopes derived from one or more of the Gag, Pol and Env polyprotein sequences of HIV. The epitope sequences of the mosaic HIV Gag-Pol-Env antigens according to the invention resemble the sequences of the natural HIV antigens, but are optimized to present a broader possible array of T cell epitopes to improve coverage of epitopes found in circulating HIV sequences.

For example, to provide maximal coverage of potential T-cell epitopes, mosaic Gag, Pol and Env antigens are designed to provide optimal coverage of one or more HIV clades. Sequence Database in silico recombinant sequences of fragments of 9 contiguous amino acids (9-mers) are selected that resemble real proteins and that maximize the number of 9-mer sequence matches between vaccine candidates and the global database. The mosaic Gag, Pol and Env antigens have similar domain structure to natural antigens and consist entirely of natural sequences with no artificial junctions. The Pol antigens can contain mutants to eliminate catalytic activity. The monomeric Env gp140 mosaic antigens can contain point mutations to eliminate cleavage and fusion activity.

In one embodiment, a mosaic HIV Gag-Pol-Env antigen according to the invention is a mosaic HIV Gag antigen with epitopes derived from the sequences of gag gene products; a mosaic HIV Pol antigen with epitopes derived from the sequences of pol gene products; or a mosaic HIV Env antigen with epitopes derived from the sequences of env gene products.

In another embodiment, a mosaic HIV Gag-Pol-Env antigen according to the invention comprises a combination of epitopes derived from sequences of gag, pol, and/or env gene products. Illustrative and non-limiting examples include mosaic Gag-Pol antigens with epitopes derived from the sequences of gag and pol gene products.

Examples of mosaic HIV Gag-Pol-Env antigens include those described in, e.g., US20120076812, Barouch et al., Nat Med 2010, 16:319-323 [54]; Barouch et al., Cell 155:1-9, 2013 [65], all of which are incorporated herein by reference in their entirety.

Preferably, mosaic HIV Gag-Pol-Env antigens include, but are not limited to, antigens comprising the amino acid sequences selected from the group consisting of SEQ ID NOs: 1-4 and 8, more preferably SEQ ID NO: 1, SEQ ID NO: 3 or SEQ ID NO: 4.

In view of the present disclosure, a mosaic HIV antigen can be produced using methods known in the art. See, for example, US20120076812, Fischer et al, Nat Med, 2007. 13(1): p. 100-6 [53]; Barouch et al., Nat Med 2010, 16:319-323 [54], all of which are incorporated herein by reference in their entirety.

Envelope Glycoprotein

As used herein, each of the terms “envelope glycoprotein,” “env glycoprotein,” and “Env” refers to, but is not limited to, the glycoprotein that is expressed on the surface of the envelope of HIV virions and the surface of the plasma membrane of HIV infected cells, or a fragment thereof that can induce an immune response or produce an immunity against the HIV in a subject in need thereof.

The env gene encodes gp160, which is proteolytically cleaved into gp120 and gp41. More specifically, gp160 trimerizes to (gp160)₃ and then undergoes cleavage into the two noncovalently associated fragments gp120 and gp41. Viral entry is subsequently mediated by a trimer of gp120/gp41 heterodimers. Gp120 is the receptor binding fragment, and binds to the CD4 receptor on a target cell that has such a receptor, such as, e.g., a T-helper cell. Gp41, which is non-covalently bound to gp120, is the fusion fragment and provides the second step by which HIV enters the cell. Gp41 is originally buried within the viral envelope, but when gp120 binds to a CD4 receptor, gp120 changes its conformation causing gp41 to become exposed, where it can assist in fusion with the host cell. Gp140 is the uncleaved ectodomain of trimeric gp160, i.e., (gp160)₃, that has been used as a surrogate for the native state of the cleaved, viral spike.

According to one embodiment of the invention, env glycoproteins (e.g., gp160, gp140, gp120, or gp41), preferably stabilized trimeric gp140 protein, can be administered for priming or boosting immunizations to enhance the immunity induced by expression vectors alone.

As used herein, each of the terms “stabilized trimeric gp140 protein” and “stabilized trimer of gp140” refers to a trimer of gp140 polypeptides that includes a polypeptide sequence that increases the stability of the trimeric structure. The gp140 polypeptides can have, or can be modified to include a trimerization domain that stabilizes trimers of gp140. Examples of trimerization domains include, but are not limited to, the T4-fibritin “foldon” trimerization domain; the coiled-coil trimerization domain derived from GCN4 [66]; and the catalytic subunit of E. coli aspartate transcarbamoylase as a trimer tag [67].

In a particular embodiment of the invention, a stabilized trimeric gp140 protein comprises the amino acid sequence of SEQ ID NO: 5 (clade C gp140 protein).

According to one embodiment of the invention, a stabilized trimeric gp140 protein can be administered as a boosting immunization or as a component of a boosting immunization together with viral expression vectors. Preferably, the stabilized trimeric gp140 protein is a clade C gp140 protein. A clade C trimeric gp140 protein is able to induce potent neutralizing antibody responses against a set of HIV-1 variants from different clades and with different neutralization sensitivities in guinea pigs [68, 60].

According to another embodiment of the invention, the “envelope glycoprotein” is a mosaic envelope protein comprising multiple epitopes derived from one or more of Env polyprotein sequences of one or more HIV clades. For example, as used herein a “gp140 protein” can be a “mosaic gp140 protein” that contains multiple epitopes derived from one or more gp140 protein sequences of one or more HIV clades.

In a particular embodiment of the invention, a mosaic gp140 protein is a stabilized trimer of mosaic gp140 comprising the amino acid sequence of SEQ ID NO: 6.

In certain embodiments, two gp140 proteins are administered to the same subject, preferably a clade C gp140 having the amino acid sequence of SEQ ID NO: 5 and a mosaic gp140 having the amino acid sequence of SEQ ID NO: 6. These can be together in one pharmaceutical composition, preferably administered together with aluminum phosphate adjuvant. A preferred dose for the total amount of gp140 for administration to humans is between about 125 and 350 μg, preferably about 250 μg. If clade C gp140 and mosaic gp140 are both administered, a suitable dose would for instance be about 125 μg of each protein, to a total of 250 μg of gp140 protein for an administration to humans.

An isolated gp140 protein can be co-delivered with an adenovirus expression vector or MVA expression vector. According to a preferred embodiment, a gp140 protein and Ad26 or MVA are administered separately, as two distinct formulations. Alternatively, a gp140 protein can be administered with Ad26 or MVA together in a single formulation. Simultaneous administration or co-delivery can take place at the same time, within one hour, or within the same day. Furthermore, a gp140 protein can be administered in an adjuvanted formulation. Suitable adjuvants can be, for example, aluminum phosphate or a saponin-based adjuvant.

Antigenic polypeptides can be produced and isolated using any method known in the art in view of the present disclosure. For example, an antigenic polypeptide can be expressed from a host cell, preferably a recombinant host cell optimized for production of the antigenic polypeptide. According to an embodiment of the invention, a recombinant gene is used to express a gp140 protein containing mutations to eliminate cleavage and fusion activity, preferably an optimized gp140 protein with increased breadth, intensity, depth, or longevity of the antiviral immune response (e.g., cellular or humoral immune responses) generated upon immunization (e.g., when incorporated into a composition of the invention, e.g., vaccine of the invention) of a subject (e.g., a human). The optimized gp140 protein can also include cleavage site mutation(s), a factor Xa site, and/or a foldon trimerization domain. A leader/signal sequence can be operably linked to the N-terminal of an optimized gp140 protein for maximal protein expression. The leader/signal sequence is usually cleaved from the nascent polypeptide during transport into the lumen of the endoplasmic reticulum. Any leader/signal sequence suitable for a host cell of interest can be used. An exemplary leader/signal sequence comprises the amino acid sequence of SEQ ID NO:7.

In a preferred embodiment of the invention, the isolated antigenic polypeptide is a stabilized trimeric gp140 as those described in Nkolola et al 2010, J. Virology 84(7): 3270-3279 [68]; Kovacs et al, PNAS 2012, 109(30):12111-6 [60], WO 2010/042942 and WO 2014/107744, all of which are incorporated by reference in their entirety.

Adenoviruses

An adenovirus according to the invention belongs to the family of the Adenoviridae, and preferably is one that belongs to the genus Mastadenovirus. As used herein, the notation “rAd” means recombinant adenovirus, e.g., “rAd26” refers to recombinant human adenovirus 26.

According to the methods of the invention, an adenovirus is a human adenovirus serotype 26. An advantage of rAd26 is a low seroprevalence and/or low pre-existing neutralizing antibody titers in the human population. Preparation of rAd26 vectors is described, for example, in WO 2007/104792 and in Abbink et al., (2007) Virol 81(9): 4654-63 [11], both of which are incorporated by reference herein in their entirety. Exemplary genome sequences of Ad26 are found in GenBank Accession EF 153474 and in SEQ ID NO: 1 of WO 2007/104792.

Thus, the vectors that can be used in an embodiment of the invention comprise an Ad26 capsid protein (e.g., a fiber, penton or hexon protein). One of ordinary skill in the art will recognize that it is not necessary that an entire Ad26 capsid protein be used in the vectors of the invention. Thus, chimeric capsid proteins that include at least a part of an Ad26 capsid protein can be used in the vectors of the invention. The vectors according to embodiments of the invention can also comprise capsid proteins in which the fiber, penton, and hexon proteins are each derived from a different serotype, so long as at least one capsid protein is derived from Ad26. In preferred embodiments, the fiber, penton and hexon proteins are each derived from Ad26.

In certain embodiments the recombinant adenovirus vector useful in the invention is derived mainly or entirely from Ad26 (i.e., the vector is rAd26). In preferred embodiments, the adenovirus is replication deficient, e.g., because it contains a deletion in the E1 region of the genome. For the adenoviruses of the invention, being derived from Ad26, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g. Havenga, et al., 2006, J Gen Virol 87: 2135-43 [61]; WO 03/104467). However, such adenoviruses will not be capable of replicating in non-complementing cells that do not express the E1 genes of Ad5.

In certain embodiments, the adenovirus is a human adenovirus of serotype 26, with a deletion in the E1 region into which the nucleic acid encoding the one or more HIV antigenic polypeptides has been cloned, and with an E4 orf6 region of Ad5. The preparation of recombinant adenoviral vectors is well known in the art. Preparation of rAd26 vectors is described, for example, in WO 2007/104792 and in Abbink et al., (2007) Virol 81(9): 4654-63 [11]. Exemplary genome sequences of Ad26 are found in GenBank Accession EF 153474 and in SEQ ID NO:1 of WO 2007/104792. In an embodiment of the invention, the vectors useful for the invention include those described in WO2012/082918, the disclosure of which is incorporated herein by reference in its entirety.

Typically, a vector useful in the invention is produced using a nucleic acid comprising the entire recombinant adenoviral genome (e.g., a plasmid, cosmid, or baculovirus vector). Thus, the invention also provides isolated nucleic acid molecules that encode the adenoviral vectors of the invention. The nucleic acid molecules of the invention can be in the form of RNA or in the form of DNA obtained by cloning or produced synthetically. The DNA can be double-stranded or single-stranded.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding an antigenic polypeptide (usually linked to a promoter) within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines include, for example, PER.C6, 911, 293, and E1 A549.

As noted above, a wide variety of HIV antigenic polypeptides can be expressed in the vectors. If required, the heterologous gene encoding the HIV antigenic polypeptides can be codon-optimized to ensure proper expression in the treated host (e.g., human). Codon-optimization is a technology widely applied in the art. Typically, the heterologous gene is cloned into the E1 and/or the E3 region of the adenoviral genome.

The heterologous HIV gene can be under the control of (i.e., operably linked to) an adenovirus-derived promoter (e.g., the Major Late Promoter), or can be under the control of a heterologous promoter. Examples of suitable heterologous promoters include the cytomegalovirus (CMV) promoter and the Rous Sarcoma virus (RSV) promoter. Preferably, the promoter is located upstream of the heterologous gene of interest within an expression cassette.

As noted above, the adenovirus vectors useful for the invention can encode a wide variety of HIV antigenic polypeptides known to those of skill in the art, including but not limited to, the antigenic polypeptides discussed herein. In preferred embodiments the one or more rAd26 vectors together encode HIV antigenic polypeptides having amino acid sequences of SEQ ID NOs: 1, 3, and 4. In certain embodiments, the one or more rAd26 vectors further encode HIV antigenic polypeptide having SEQ ID NO: 8.

In a preferred embodiment of the invention, the adenovirus vectors are rAd26 vector, such as that described in Abbink, J Virol, 2007. 81(9): p. 4654-63 [11], which is incorporated herein by reference.

MVA Vectors

MVA vectors useful for the invention utilize attenuated virus derived from Modified Vaccinia Ankara virus, which is characterized by the loss of their capabilities to reproductively replicate in human cell lines. The MVA vectors can express any of the HIV antigenic polypeptides known to those of skill in the art, including but not limited to the antigenic polypeptides discussed herein.

MVA has been generated by more than 570 serial passages on chicken embryo fibroblasts of the dermal vaccinia strain Ankara [Chorioallantois vaccinia virus Ankara virus, CVA; for review see Mayr et al. (1975), Infection 3, 6-14 [74]] that was maintained in the Vaccination Institute, Ankara, Turkey for many years and used as the basis for vaccination of humans. However, due to the often severe post-vaccination complications associated with vaccinia viruses, there were several attempts to generate a more attenuated, safer smallpox vaccine.

During the period of 1960 to 1974, Prof. Anton Mayr succeeded in attenuating CVA by over 570 continuous passages in CEF cells [74]. It was shown in a variety of animal models that the resulting MVA was avirulent [75]. As part of the early development of MVA as a pre-smallpox vaccine, there were clinical trials using MVA-517 in combination with Lister Elstree [77, 78] in subjects at risk for adverse reactions from vaccinia. In 1976, MVA derived from MVA-571 seed stock (corresponding to the 571^{st} passage) was registered in Germany as the primer vaccine in a two-stage parenteral smallpox vaccination program. Subsequently, MVA-572 was used in approximately 120,000 Caucasian individuals, the majority children between 1 and 3 years of age, with no reported severe side effects, even though many of the subjects were among the population with high risk of complications associated with vaccinia [76]. MVA-572 was deposited at the European Collection of Animal Cell Cultures as ECACC V94012707.

As a result of the passaging used to attenuate MVA, there are a number of different strains or isolates, depending on the number of passages conducted in CEF cells. For example, MVA-572 was used in a small dose as a pre-vaccine in Germany during the smallpox eradication program, and MVA-575 was extensively used as a veterinary vaccine. MVA as well as MVA-BN lacks approximately 15% (31 kb from six regions) of the genome compared with ancestral CVA virus. The deletions affect a number of virulence and host range genes, as well as the gene for Type A inclusion bodies. MVA-575 was deposited on Dec. 7, 2000, at the European Collection of Animal Cell Cultures (ECACC) under Accession No. V00120707. The attenuated CVA-virus MVA (Modified Vaccinia Virus Ankara) was obtained by serial propagation (more than 570 passages) of the CVA on primary chicken embryo fibroblasts.

Even though Mayr et al. demonstrated during the 1970s that MVA is highly attenuated and avirulent in humans and mammals, certain investigators have reported that MVA is not fully attenuated in mammalian and human cell lines since residual replication might occur in these cells [79, 80; U.S. Pat. No. 5,185,146; 81]. It is assumed that the results reported in these publications have been obtained with various known strains of MVA, since the viruses used essentially differ in their properties, particularly in their growth behaviour in various cell lines. Such residual replication is undesirable for various reasons, including safety concerns in connection with use in humans.

Strains of MVA having enhanced safety profiles for the development of safer products, such as vaccines or pharmaceuticals, have been developed, for example by Bavarian Nordic. MVA was further passaged by Bavarian Nordic and is designated MVA-BNA. A representative sample of MVA-BN was deposited on Aug. 30, 2000 at the European Collection of Cell Cultures (ECACC) under Accession No. V00083008. MVA-BN is further described in WO 02/42480 (US 2003/0206926) and WO 03/048184 (US 2006/0159699), both of which are incorporated by reference herein in their entirety.

“Derivatives” or “variants” of MVA refer to viruses exhibiting essentially the same replication characteristics as MVA as described herein, but exhibiting differences in one or more parts of their genomes. For example, MVA-BN as well as a derivative or variant of MVA-BN fails to reproductively replicate in vivo in humans and mice, even in severely immune suppressed mice. More specifically, MVA-BN or a derivative or variant of MVA-BN has preferably also the capability of reproductive replication in chicken embryo fibroblasts (CEF), but no capability of reproductive replication in the human keratinocyte cell line HaCat [82], the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, a derivative or variant of MVA-BN has a virus amplification ratio at least two fold less, more preferably three-fold less than MVA-575 in Hela cells and HaCaT cell lines. Tests and assays for these properties of MVA variants are described in WO 02/42480 (US 2003/0206926) and WO 03/048184 (US 2006/0159699).

The term “not capable of reproductive replication” or “no capability of reproductive replication” is, for example, described in WO 02/42480, which also teaches how to obtain MVA having the desired properties as mentioned above. The term applies to a virus that has a virus amplification ratio at 4 days after infection of less than 1 using the assays described in WO 02/42480 or in U.S. Pat. No. 6,761,893, both of which are incorporated by reference herein in their entirety.

The term “fails to reproductively replicate” refers to a virus that has a virus amplification ratio at 4 days after infection of less than 1. Assays described in WO 02/42480 or in U.S. Pat. No. 6,761,893 are applicable for the determination of the virus amplification ratio.

The amplification or replication of a virus is normally expressed as the ratio of virus produced from an infected cell (output) to the amount originally used to infect the cell in the first place (input), and is referred to as the “amplification ratio”. An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

The advantages of MVA-based vaccine include their safety profile as well as availability for large scale vaccine production. Furthermore, in addition to its efficacy, the feasibility of industrial scale manufacturing can be beneficial. Additionally, MVA-based vaccines can deliver multiple heterologous antigens and allow for simultaneous induction of humoral and cellular immunity.

MVA vectors useful for the invention can be prepared using methods known in the art, such as those described in WO/2002/042480, WO/2002/24224, US20110159036, U.S. Pat. No. 8,197,825, etc., the relevant disclosures of which are incorporated herein by references.

In another aspect, replication deficient MVA viral strains can also be suitable for use in the invention, such as strains MVA-572 and MVA-575, or any other similarly attenuated MVA strain. Also suitable can be a mutant MVA, such as the deleted chorioallantois vaccinia virus Ankara (dCVA). A dCVA comprises del I, del II, del III, del IV, del V, and del VI deletion sites of the MVA genome. The sites are particularly useful for the insertion of multiple heterologous sequences. The dCVA can reproductively replicate (with an amplification ratio of greater than 10) in a human cell line (such as human 293, 143B, and MRC-5 cell lines), which then enable the optimization by further mutation useful for a virus-based vaccination strategy (see WO 2011/092029).

In a preferred embodiment of the invention, the MVA vector(s) comprise a nucleic acid that encodes one or more antigenic HIV proteins, such as the HIV mosaic antigen. In preferred embodiments, the one or more MVA vectors together encode one or more HIV antigenic polypeptides comprising the amino acid sequences selected from the group consisting of SEQ ID NOs: 1-4, preferably at least three HIV antigenic polypeptides having the amino acid sequences of SEQ ID NOs: 1, 3 and 4, and more preferably encode four HIV antigenic polypeptides having the amino acid sequences of SEQ ID NO: 1, SEQ ID NO: 2, SEQ ID NO: 3, and SEQ ID NO: 4. In certain embodiments, they may also encode HIV antigenic polypeptide encoding SEQ ID NO: 8.

Nucleic acid sequences encoding the HIV antigenic protein can be inserted into one or more intergenic regions (IGR) of the MVA. In certain embodiments, the IGR is selected from IGR07/08, IGR 44/45, IGR 64/65, IGR 88/89, IGR 136/137, and IGR 148/149. In certain embodiments, less than 5, 4, 3, or 2 IGRs of the recombinant MVA comprise heterologous nucleotide sequences encoding antigenic determinants of a HIV, such as a mosaic antigen and/or a further HIV antigenic polypeptide. The heterologous nucleotide sequences can, additionally or alternatively, be inserted into one or more of the naturally occurring deletion sites, in particular into the main deletion sites I, II, III, IV, V, or VI of the MVA genome. In certain embodiments, less than 5, 4, 3, or 2 of the naturally occurring deletion sites of the recombinant MVA comprise heterologous nucleotide sequences encoding antigenic determinants of a HIV envelope glycoprotein and/or a further HIV protein.

The number of insertion sites of MVA comprising heterologous nucleotide sequences encoding antigenic determinants of a HIV protein can be 1, 2, 3, 4, 5, 6, 7, or more. In certain embodiments, the heterologous nucleotide sequences are inserted into 4, 3, 2, or fewer insertion sites. Preferably, two insertion sites are used. In certain embodiments, three insertion sites are used. Preferably, the recombinant MVA comprises at least 2, 3, 4, 5, 6, or 7 genes inserted into 2 or 3 insertion sites.

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual (2nd Ed.) [83], and techniques for the handling and manipulation of viruses are described in Virology Methods Manual [B. W. J. Mahy et al. (eds.), Academic Press (1996)]. Similarly, techniques and know-how for the handling, manipulation and genetic engineering of MVA are described in Molecular Virology: A Practical Approach [A. J. Davison & R. M. Elliott (Eds.), The Practical Approach Series, IRL Press at Oxford University Press, Oxford, UK (1993)(see, e.g., Chapter 9: Expression of genes by Vaccinia virus vectors)] and Current Protocols in Molecular Biology [John Wiley & Son, Inc. (1998)(see, e.g., Chapter 16, Section IV: Expression of proteins in mammalian cells using vaccinia viral vector)].

For the generation of the various recombinant MVAs disclosed herein, different methods can be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture such as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter. Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome cloned as a bacterial artificial chromosome (BAC) and a linear foreign sequence flanked with DNA sequences homologous to sequences flanking the desired site of integration in the vaccinia virus genome.

The heterologous HIV gene, e.g., nucleic acid encoding one or more HIV antigenic polypeptides, can be under the control of (i.e., operably linked to) one or more poxvirus promoters. In certain embodiments, the poxvirus promoter is a Pr7.5 promoter, a hybrid early/late promoter, or a PrS promoter, a PrS5E promoter, a synthetic or natural early or late promoter, or a cowpox virus ATI promoter.

In a preferred embodiment of the invention, the MVA vectors express polyvalent mosaic Env/Gag/Pol antigens, such as those described in Barouch et al., Nat Med 2010, 16:319-323 [54]; Barouch et al., Cell 155:1-9, 2013 [65], all of which are incorporated herein by reference in their entirety. According to embodiments of the invention, MVA vectors can express any of the antigenic polypeptides described herein including, but not limited to, HIV mosaic antigens, such as HIV mosaic Gag-Pol-Env antigens.

Immunogenic Compositions

As used herein, “an immunogenically effective amount” or “immunologically effective amount” means an amount of a composition sufficient to induce a safe and effective immune response in a human subject in need thereof.

According to embodiments of the invention, an immunogenically effective amount of the priming composition or the second boosting composition when used with reference to total amount of Ad26 vectors in the composition can range from about 5×10⁹ to about 1×10¹¹ viral particles, for example 5×10⁹, 10¹⁰, 5×10¹⁰ or 10¹¹ viral particles. In certain embodiments, when 2, 3, or 4 adenoviral vectors are present in a composition, they are present at a 1:1, 1:1:1 or 1:1:2, or 1:1:1:1 ratio.

According to embodiments of the invention, when used with reference to the total amount of the at least one isolated HIV envelope glycoprotein in the first boosting composition, such as the isolated gp140 protein having the amino acid sequence of SEQ ID NO: 5, an immunogenically effective amount can range from, e.g. about 125 μg to 350 μg, e.g. about 125, 150, 200, 250, 300, 350 μg. In certain embodiments, the first boosting composition comprises two isolated HIV envelope gp140 proteins, one clade C gp140 having the amino acid sequence of SEQ ID NO: 5 and one mosaic gp140 having the amino acid sequence of SEQ ID NO: 6, each one for instance present in about 125 μg per administration to a total of about 250 μg.

According to embodiments of the invention, an immunogenically effective amount of the second boosting composition when used with reference to total amount of MVA vectors in the composition can range from about 10⁷ to about 10⁹ plaque-forming units (pfu), for example, about 10⁷, 5×10⁷, 10⁸, 5×10⁸, or 10⁹.

It is possible to administer an immunogenically effective amount to a subject, and subsequently administer another dose of an immunogenically effective amount to the same subject, in a so-called prime-boost regimen. This general concept of a prime-boost regimen is well known to the skill person in the vaccine field. Further booster administrations can optionally be added to the regimen, as needed.

An immunogenically effective amount can be administered in a single step (such as a single injection), or multiple steps (such as multiple injection), or in a single composition or multiple compositions.

Immunogenic compositions are compositions comprising an immunogenically effective amount of purified or partially purified adenovirus or MVA vectors for use in the invention. Said compositions can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

The preparation and use of immunogenic compositions are well known to those of ordinary skill in the art. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can also be included.

The compositions of the invention can comprise other HIV-1 antigens or the priming or boosting immunizations can comprise other antigens. The other antigens used in combination with the adenovirus vectors of the invention are not critical to the invention and can be, for example, HIV-1 antigens and nucleic acids expressing them.

The immunogenic compositions useful in the invention can comprise adjuvants. Adjuvants suitable for co-administration in accordance with the invention should be ones that are potentially safe, well tolerated and effective in people including QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Aluminium salts (e.g. alumunim phosphate, e.g. AdjuPhos), Adjuplex, and MF59.

A preferred adjuvant for administration together with isolated HIV envelope glycoprotein is aluminum phosphate. According to embodiments of the invention, when used with reference to the total amount of aluminum phosphate in the first boosting composition, the amount of aluminum phosphate can range from, e.g. about 10 mg to about 1000 mg, e.g. about 200 mg to 650 mg, e.g. about 200, 250, 300, 350, 400, 425, 450, 475, 500, 550, or 600 mg.

The compositions of the invention can comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., intramuscular, subcutaneous, oral, intravenous, cutaneous, intramucosal (e.g., gut), intranasal or intraperitoneal routes.

The ability to induce or stimulate an anti-HIV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [³H] thymidine uptake), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

According to embodiments of the invention, upon administration to a subject, an expression vector, such as a recombinant adenovirus vector or recombinant MVA vector, expresses an immunogenic polypeptide. Any of the antigenic polypeptides described herein can be encoded by an expression vector and administered to a subject in a method of the invention. The expressed immunogenic polypeptide is presented to the immune system of the subject, thereby inducing the required response to produce immunity, or induce an immune response to treat or prevent a disease or infection. For example, the response can be the production of antibodies specific to the immunogenic polypeptide.

Preferably, upon administration to a subject, an expression vector expresses a mosaic HIV Gag-Pol-Env antigen. Presentation of a mosaic HIV Gag-Pol-Env antigen according to the invention to the immune system of a subject can induce the production of antibodies specific to the HIV gag, pol, and/or env gene products, depending on the sequence composition of the expressed mosaic HIV antigen.

Vaccine Combination

A vaccine combination useful for inducing an immune response against a human immunodeficiency virus (HIV) in a subject in need thereof, can comprise:

(i) a priming composition comprising one or more Ad26 vectors encoding at least three HIV antigenic polypeptides having the amino acid sequences of SEQ ID NO: 1, SEQ ID: NO 3, and SEQ ID NO: 4, and a pharmaceutically acceptable carrier;
(ii) a first boosting composition comprising an isolated HIV envelope glycoprotein having the amino acid sequence of SEQ ID NO: 5, an aluminum phosphate adjuvant and a pharmaceutically acceptable carrier;
(iii) a second boosting composition comprising one or more Ad26 vectors encoding the at least three HIV antigenic polypeptides and a pharmaceutically acceptable carrier, or a second alternative boosting composition comprising one or more MVA vectors encoding the at least three HIV antigenic polypeptides and an additional HIV antigenic polypeptide comprising the amino acid sequence of SEQ ID NO:2, and a pharmaceutically acceptable carrier.

The priming composition is for priming immunization. The first, second or second alternative boosting compositions are for boosting immunization.

In one embodiment of the invention, the first boosting composition further comprises another HIV antigenic peptide, preferably a mosaic HIV envelope glycoprotein, such as a mosaic HIV gp140 protein, such as that comprising the amino acid sequence of SEQ ID NO: 6.

In embodiments of the invention, one or more rAd26 vectors are used for the priming immunization, and one or more rAd26 vectors, together with an isolated HIV antigenic polypeptide, such as an HIV envelope protein, preferably a stabilized trimeric gp140 protein, are used for the boosting immunization. The adenovirus vectors used for boosting immunization can encode the same antigenic proteins as those encoded by the adenovirus vectors used for priming immunization.

In other embodiments of the invention, one or more rAd26 vectors are used for the priming immunization, and one or more MVA vectors, together with an isolated HIV antigenic polypeptide, such as an HIV envelope protein, preferably a stabilized trimeric gp140 protein, are used for the boosting immunization. The MVA vectors used for boosting immunization can encode the same antigenic proteins as those encoded by the adenovirus vectors used for priming immunization. The MVA vectors can also encode additional antigenic peptides, such as that having the amino acid sequence of SEQ ID NO: 2, that are not encoded by the adenovirus vectors used for priming immunization.

In a particularly preferred embodiment of the invention, the priming composition comprises at least three rAd26 vectors encoding at least three mosaic HIV proteins having the amino acid sequences of SEQ ID NO: 1, SEQ ID NO: 3 and SEQ ID NO: 4, respectively; the first boosting composition comprises an isolated stabilized trimer of HIV gp140 having the amino acid sequence of SEQ ID NO: 5; and the second boosting composition comprises MVA vectors encoding four mosaic HIV antigenic proteins having the amino acid sequences of SEQ ID NOs: 1 to 4.

According to embodiments of the invention, the first composition can comprise one rAd26 vector, or more than one rAd26 vector. In certain embodiments, the first composition comprises more than one rAd26 vector, such as one, two, three, or four, etc. rAd26 vectors. The one or more rAd26 vectors can express the same or different HIV antigenic polypeptides. Each of the expression vectors can express one HIV antigenic polypeptide sequence, or more than one HIV antigenic polypeptide sequence. As an illustrative and non-limiting preferred example, the first composition can comprise three rAd26 vectors, each expressing a different HIV antigenic polypeptide, preferably SEQ ID NOs: 1, 3, and 4. The first composition can also comprise more than three rAd26 vectors, such as four rAd26 vectors, encoding additional HIV antigenic polypeptide(s), such as one having the amino acid sequence of SEQ ID NO: 8. In certain embodiments, the one or more Ad26 vectors express four HIV antigenic polypeptides, respectively having amino acid sequences of SEQ ID NOs: 1, 3, 4 and 8.

According to embodiments of the invention, the one or more additional expression vectors can be one expression vector, or more than expression vector, such as two, three, four or more expression vectors. The one or more additional expression vectors can express the same or different antigenic polypeptides. Each of the one more additional expression vectors can express one antigenic polypeptide sequence, or multiple antigenic polypeptide sequences. As an illustrative and non-limiting example, two additional expression vectors are used, preferably MVA vectors, with each MVA vector encoding a different mosaic HIV antigen sequence, such as mosaic HIV Gag-Pol-Env antigen sequences selected from the group consisting of SEQ ID NOs: 1-4 and 8. In certain of such embodiments of the invention, one MVA vector encodes HIV antigenic polypeptides comprising SEQ ID NOs: 1 and 3, and the other MVA vector encodes HIV antigenic polypeptides comprising SEQ ID NOs: 4 and 8.

The vaccine combination according to embodiments of the invention is effective to induce an immune response against one or multiple clades of HIV.

Method for Inducing Protective Immunity Against HIV Infection

The vaccine combinations according to embodiments of the invention can be used in a method of the invention described herein.

According to embodiments of the invention, “inducing an immune response” when used with reference to the methods described herein encompasses providing protective immunity and/or vaccinating a subject against an infection, such as a HIV infection, for prophylactic purposes, as well as causing a desired immune response or effective in a subject in need thereof against an infection, such as a HIV infection, for therapeutic purposes. Preferably, the methods of the invention are for prophylactic purposes, such as for providing protective immunity. Preferably the subject to which the compositions is administered is a human subject uninfected by HIV.

Embodiments of the isolated antigenic polypeptides, expression vectors, additional expression vectors, antigenic polypeptide encoded by the expression vectors, etc. that can be used in the methods of the invention are discussed in detail above and in the illustrative examples below.

In one embodiment of the disclosed methods, one or more rAd26 vectors encoding one or more HIV antigenic polypeptides are used to prime the immune response. One or more isolated HIV antigenic polypeptides can be used together with the one or more adenovirus vectors for the boosting immunization. The priming immunization can be administered multiple times, for example, initial priming administration at time 0, followed by another priming administration about 10-14 weeks, such as 10, 11, 12, 13 or 14 weeks, after the initial priming administration. One or more isolated HIV antigenic polypeptides together with one or more additional rAd26 or MVA vectors encoding one or more additional HIV antigenic polypeptides are used to boost the immune response. The boosting immunization can also be administered multiple times, for example, first at about 22-26 weeks, such as 22, 23 24, 25, or 26 weeks, after the initial priming administration, preferably followed by another boosting administration at about 42-60 weeks, such as 42, 43, 44, 45, 46, 47, 48, 49, 50, 51, 52, 53, 54, 55, 56, 57, 58, 59 or 60 weeks after the initial priming administration. The immune response induced by the immunization can be monitored.

Embodiments of the disclosed methods also contemplate shorter prime-boost regimens, meaning that the final boosting immunization is administered about 22-26 weeks after the initial priming administration, and the priming immunization can be administered at week 0, and re-administered at about 10-14 weeks. The boosting immunization can also be administered multiple times following the priming administration. In certain embodiments, one or more isolated HIV antigenic polypeptides is administered together with the one or more adenovirus vectors for the boosting immunization.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 2-3 weeks or 4 weeks, or 8 weeks, or 16 weeks, or 20 weeks, or 24 weeks, or 28 weeks, or 30 weeks or 32 weeks or one to two years after administration of the priming composition.

In certain embodiments herein, at least part of the immune responses induced by the compositions in the regimens disclosed herein are persistent immune responses. An immune response is considered persistent as used herein when the immune response is still significantly above background (e.g., the immune responses measured when placebo is administered instead of the priming and boosting compositions as described herein, or the immune response measured just before the first administration of the priming composition) at least 26 weeks, preferably at least 36 weeks, more preferably at least 48 weeks after the last administration of the boosting compositions. In certain embodiments this can be at least 96 weeks after administration of the first priming composition. In preferred embodiments, the immune response is not decreased by two orders of magnitude at the time point of at least 26 weeks, preferably at least 36 weeks, more preferably at least 48 weeks after the last administration of the boosting compositions, as compared to the immune response as measured four weeks after the last administration of the boosting compositions. Such persistent immune responses were observed in humans upon administration of the vaccine components in the regimens disclosed herein. Also this aspect can be seen as a surprising result, given previous reports describing rapid waning of immune responses to undetectable levels of another HIV vaccine candidate in human trials [9]. The immune responses generated using the components and regimens described herein can last longer than 48 weeks after the last administration of the booster compositions, which can be determined by following the immunized subjects and measuring the immune responses at later time points, e.g. at one, two or more years after the last administration of the booster compositions. It is also possible to administer one or more further booster compositions at later time points. In certain embodiments, the persistent immune response comprises a persistent humoral immune response against HIV envelope glycoprotein of at least Clade C. In certain embodiments, the humoral response can also be against HIV envelope glycoprotein of other clades, e.g. clade A, B, or mosaic envelope glycoproteins. In certain embodiments, the persistent immune response comprises a persistent cellular immune response. In certain embodiments, the persistent immune response is observed at a response rate of at least 50%, at least 60%, at least 70%, at least 80%, at least 90%, at least 95%, or about 100% of the subjects to which the priming and boosting compositions were administered according to the regimens described herein.

The adenovirus vectors used in the methods disclosed herein include a rAd26 vector. In one exemplary embodiment, a rAd26 vector is used to prime the immune response, and an MVA vector together with an isolated antigenic polypeptide is used to boost the immune response, or vice versa. In a preferred embodiment, a rAd26 vector is used to prime the immune response, and a rAd26 vector together with an isolated antigenic polypeptide is used to boost the immune response.

In one or more embodiments of the described method, a plurality of rAd26 vectors are used to prime the immune response, and a plurality of isolated antigenic proteins, together with a plurality of rAd26 or MVA vectors, are used to boost the immune response.

In certain embodiments, a first boosting immunization is administered 10-36 weeks after the last priming, more preferably 12-24 weeks after priming.

The antigens in the respective priming and boosting compositions (however many boosting compositions are employed) need not be identical, but should share antigenic determinants or be substantially similar to each other.

Administration of the immunogenic compositions comprising the expression vectors and/or antigenic polypeptides is typically intramuscular or subcutaneous. However other modes of administration such as intravenous, cutaneous, intradermal or nasal can be envisaged as well. Intramuscular administration of the immunogenic compositions can be achieved by using a needle to inject a suspension of the expression vectors, e.g. adenovirus and/or MVA vectors, and/or antigenic polypeptides. An alternative is the use of a needleless injection device to administer the composition (using, e.g., Biojector™) or a freeze-dried powder containing the vaccine.

For intravenous, cutaneous or subcutaneous injection, or injection at the site of affliction, the vector will be in the form of a parenterally acceptable aqueous solution which is pyrogen-free and has suitable pH, isotonicity and stability. Likewise, the isolated antigenic polypeptide will be in the form of a parenterally acceptable solution having a suitable pH, isotonicity, and stability. Those of ordinary skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

Typically, administration of the vaccine compositions according to embodiments of the invention will have a prophylactic aim to generate an immune response against an HIV antigen before infection or development of symptoms.

The immunogenic compositions containing the expression vectors, e.g., adenovirus vectors and/or MVA vectors, and antigenic polypeptides are administered to a subject, giving rise to an anti-HIV immune response in the subject. An amount of a composition sufficient to induce a detectable immune response is defined to be an “immunogenically effective dose.” As shown in the Examples below, the immunogenic compositions of the invention induce a humoral as well as a cell-mediated immune response.

The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. ed., 1980.

Following production of adenovirus and MVA vectors and optional formulation of such particles into compositions, the vectors can be administered to a human according to embodiments of the invention.

In one exemplary regimen, the adenovirus or MVA vector is administered (e.g., intramuscularly) in the range of from about 100 μl to about 10 ml of saline solution containing concentrations of from about 10⁴ to 10¹² virus particles/ml. Typically, the adenovirus or MVA vector is administered in an amount of about 10⁹ to about 10¹² viral particles (vp) to a human subject during one administration, more typically from about 10¹⁰ to about 10¹² vp. The initial vaccination is followed by a boost as described above. The isolated HIV antigenic polypeptide can for instance be administered ranging from about 0.001 to 30 mg/kg body weight. The skilled artisan will appreciate that certain factors may influence the dosage required to effectively treat a subject, including but not limited to the severity of the disease or disorder, previous treatments, the general health and/or age of the subject, and other diseases present.

The composition can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration.

The compositions of the invention can be administered alone or in combination with other treatments, either simultaneously or sequentially depending upon the condition to be treated, and other factors that may affect the treatment.

### EMBODIMENTS

A method of inducing a safe and effective immune response against multiple clades of human immunodeficiency virus (HIV) in a human subject in need thereof, comprising:

(1) administering to the subject a priming composition comprising one or more Ad26 vectors together encoding at least three HIV antigenic polypeptides having the amino acid sequences of SEQ ID NO: 1, SEQ ID: NO 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors;
(2) administering to the subject, about 10-14 weeks after (1), the priming composition at a total dose of about 5×10⁹ to about 1×10¹¹ vp, preferably about 5×10¹⁰ vp, of the Ad26 vectors;
(3) administering to the subject, about 22-26 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 125 μg to 350 μg, preferably about 250 μg, of the at least one isolated HIV envelope glycoprotein;
(4) administering to the subject, together with (3), a second boosting composition comprising one or more Ad26 vectors together encoding the at least three HIV antigenic polypeptides and a pharmaceutically acceptable carrier, at a total dose of about 5×10⁹ to about 1×10¹¹ vp, preferably about 5×10¹⁰ vp, of the Ad26 vectors; or
administering to the subject, together with (3), a second alternative boosting composition comprising one or more MVA vectors together encoding the at least three HIV antigenic polypeptides, and a pharmaceutically acceptable carrier, at a total dose of about 10⁷ to about 10⁹ plaque-forming units (pfu), preferably about 10⁸ pfu, of the MVA vectors,
wherein the safe and effective immune response comprises at least one selected from the group consisting of:
(a) an antibody-dependent cellular phagocytosis (ADCP) response to isolated HIV envelope glycoproteins from clades B and C at a median response rate of at least 56%, preferably also an ADCP response to isolated HIV envelope glycoproteins from clade A at a median response rate of at least 35%;
(b) a humoral immune response against HIV clades A, B and C at a median response rate of at least 90%, preferably about 100%, as measured by IgG bindings to HIV envelope glycoproteins from clades A, B and C in enzyme-linked immunosorbent assays (ELISAs); and
(c) a cellular immune response at a median response rate of at least 50%, preferably about 70%, as measured by a gammaIFN response in an ELISPOT to a potential T-cell epitopes (PTE) peptide pool.
2. The method of Embodiment 1, further comprising:
(5) administering to the subject, about 42-60 weeks after (1), the first boosting composition at a total dose of about 125 μg to 350 μg, preferably about 250 μg, of the at least one isolated HIV envelope glycoprotein; and
(6) administering to the subject, together with (5), the second boosting composition at a total dose of about 5×10⁹ to about 1×10¹¹ vp, preferably about 5×10¹⁰ vp, of the Ad26 vectors; or administering to the subject, together with (5), the second alternative boosting composition at a total dose of about 10⁷ to about 10⁹ pfu, preferably about 10⁸ pfu, of the MVA vectors.
3. The method of Embodiment 1 or 2, wherein the priming composition and the second boosting composition each comprise at least three rAd26 vectors encoding the at least three HIV antigenic polypeptides, preferably wherein the priming composition and the second boosting composition are identical.
4. The method of any one of Embodiments 1-3, wherein at the moment of step (1) the human subject is uninfected by HIV.
5. A method of inducing a safe and effective immune response against multiple clades of human immunodeficiency virus (HIV) in a human subject uninfected by HIV, comprising:
(1) administering to the subject a priming composition comprising at least three Ad26 vectors together encoding at least three HIV antigenic polypeptides having the amino acid sequences of SEQ ID NO: 1, SEQ ID: NO 3, and SEQ ID NO: 4, respectively, and a pharmaceutically acceptable carrier, in a total dose of about 5×10¹⁰ viral particles (vp) of the Ad26 vectors;
(2) administering to the subject, about 12 weeks after (1), the priming composition at a total dose of about 5×10¹⁰ vp of the Ad26 vectors;
(3) administering to the subject, about 24 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 250 μg of the at least one isolated HIV envelope glycoprotein;
(4) administering to the subject, together with (3), a second boosting composition comprising the at least three Ad26 vectors and a pharmaceutically acceptable carrier, at a total dose of about 5×10¹⁰ vp of the Ad26 vectors;
(5) administering to the subject, about 48 weeks after (1), the first boosting composition at a total dose of about 250 μg of the at least one isolated HIV envelope glycoprotein; and
(6) administering to the subject, together with (5), the second boosting composition at a total dose of about 5×10¹⁰ vp of the Ad26 vectors;
wherein the safe and effective immune response comprises at least one selected from the group consisting of:
(a) an antibody-dependent cellular phagocytosis (ADCP) response to isolated HIV envelope glycoproteins from clades B and C at a median response rate of at least 56%, preferably also an ADCP response to isolated HIV envelope glycoproteins from clade A at a median response rate of at least 35%;
(b) a humoral immune response against HIV clades A, B and C at a median response rate of at least 90%, preferably about 100%, as measured by IgG bindings to HIV envelope glycoproteins from clades A, B and C in enzyme-linked immunosorbent assays (ELISAs); and
(c) a cellular immune response at a median response rate of at least 50%, preferably about 70%, as measured by a gammaIFN response in an ELISPOT to a potential T-cell epitopes (PTE) peptide pool.
6. The method of any of Embodiments 1-5, wherein the at least three Ad26 vectors, preferably four Ad26 vectors, in the priming composition together encode a fourth HIV antigenic polypeptide having the amino acid sequence of SEQ ID NO: 8.
7. The method of any of Embodiments 1-6, wherein the at least three Ad26 vectors, preferably four Ad26 vectors, in the second boosting composition together encode a fourth HIV antigenic polypeptide having the amino acid sequence of SEQ ID NO:8, preferably wherein the priming composition and the second boosting composition are identical.
8. The method of any of Embodiments 1-4 and 6, wherein the one or more MVA vectors, preferably two MVA vectors, in the second alternative boosting composition together encode a fourth HIV antigenic polypeptide having the amino acid sequence of SEQ ID NO: 2 or preferably SEQ ID NO: 8.
9. The method of any of Embodiments 1-8, wherein the first boosting composition comprises a second isolated HIV envelope glycoprotein having the amino acid sequence of SEQ ID NO: 6, preferably in about the same amount as the isolated HIV envelope glycoprotein having the amino acid sequence of SEQ ID NO: 5.
10. The method of any of Embodiments 1-9, wherein the safe and effective immune response comprises
(a) an antibody-dependent cellular phagocytosis (ADCP) response to isolated HIV envelope glycoproteins from clades B and C at a median response rate of at least 56%, preferably also an ADCP response to isolated HIV envelope glycoproteins from clade A at a median response rate of at least 35%; and
(b) a humoral immune response against HIV clades A, B and C at a median response rate of at least 90%, preferably about 100%, as measured by IgG bindings to HIV envelope glycoproteins from clades A, B and C in enzyme-linked immunosorbent assays (ELISAs).
11. The method of any of Embodiments 1-9, wherein the safe and effective immune response comprises:
(a) an antibody-dependent cellular phagocytosis (ADCP) response to isolated HIV envelope glycoproteins from clades B and C at a median response rate of at least 56%, preferably also an ADCP response to isolated HIV envelope glycoproteins from clade A at a median response rate of at least 35%; and
(b) a cellular immune response at a median response rate of at least 50%, preferably about 70%, as measured by a gammaIFN response in an ELISPOT to a potential T-cell epitopes (PTE) peptide pool.
12. The method of any of Embodiments 1-9, wherein the safe and effective immune response comprises:
(a) a humoral immune response against HIV clades A, B and C at a median response rate of at least 90%, preferably about 100%, as measured by IgG bindings to HIV envelope glycoproteins from clades A, B and C in enzyme-linked immunosorbent assays (ELISAs); and
(b) a cellular immune response at a median response rate of at least 50%, preferably about 70%, as measured by a gammaIFN response in an ELISPOT to a potential T-cell epitopes (PTE) peptide pool.
13. The method of any of Embodiments 1-9, wherein the safe and effective immune response comprises:
(a) an antibody-dependent cellular phagocytosis (ADCP) response to isolated HIV envelope glycoproteins from clades B and C at a median response rate of at least 56%, preferably also an ADCP response to isolated HIV envelope glycoproteins from clade A at a median response rate of at least 35%;
(b) a humoral immune response against HIV clades A, B and C at a median response rate of at least 90%, preferably about 100%, as measured by IgG bindings to HIV envelope glycoproteins from clades A, B and C in enzyme-linked immunosorbent assays (ELISAs); and
(c) a cellular immune response at a median response rate of at least 50%, preferably about 70%, as measured by a gammaIFN response in an ELISPOT to a potential T-cell epitopes (PTE) peptide pool.
14. The method of any of Embodiments 1-13, wherein the ADCP response against HIV envelope glycoproteins of clade B and C is at a median response rate of about 70%, preferably about 80%.
15. The method of any of Embodiments 1-14, wherein each of the humoral immune responses against HIV envelope glycoprotein from clades A, B, and C is at a median response rate of about 100%, as measured by the ELISAs.
16. The method of any of Embodiments 1-15, wherein the cellular immune response is at a median response rate of about 70%, preferably 80% as measured by the ELISPOT.
17. The method according to any one of the preceding Embodiments, wherein the safe and effective immune response comprises a persistent humoral immune response against HIV envelope glycoprotein from at least Clade C at a response rate of at least 90%, preferably at least 95%, more preferably 100%, at 26 weeks, preferably at 36 weeks, more preferably at 48 weeks after the last administration of the boosting compositions.
18. The method according to any one of the preceding Embodiments, wherein the safe and effective immune response comprises a persistent cellular immune response against HIV envelope glycoprotein, at 26 weeks, preferably at 36 weeks, more preferably at 48 weeks after the last administration of the boosting compositions.

The following examples of the invention are to further illustrate the nature of the invention. It should be understood that the following examples do not limit the invention and the scope of the invention is to be determined by the appended claims.

### EXAMPLES

### Example 1. Study of HIV Vaccine Regimens in Non-Human Primates

An animal study was conducted to identify a multivalent HIV-1 vaccine regimen for continued advanced development. The study tested an extended vaccination schedule using two priming immunizations (at 0 weeks and 12 weeks) and a first boosting immunization (at 24 weeks). A second boosting immunization was administered at week 52. In particular, the study tested the impact of using a combination of an adenovirus or MVA vector with an envelope glycoprotein in heterologous vaccine combinations. The humoral and cellular immunological responses were tested in vaccinated non-human primates (also referred to as “NHP”).

Vaccination and Experimental Design

Rhesus monkeys (Macaca mulatta) (NHPs) were vaccinated using four different vaccine platforms with 12 animals per group (Groups II-V), in addition to two control groups (Groups I and VI) also with 12 animals each. The first control group (Group I) received primer and booster vaccines of Ad26 vectors expressing HIV-1 mosaic Env1 (SEQ ID NO: 1), mosaic GagPol1 (SEQ ID: NO 3), and mosaic GagPol2 (SEQ ID NO: 4) genes without any isolated HIV antigenic protein. The Ad26 vectors are termed “Ad26.mos1Env, Ad26.mos1Gag-Pol, and Ad26.mos2Gag-Pol, respectively, and are collectively referred to as “Ad26ₘₒₛ.” The second control group (Group VI) received only placebo (“Sham”) primer and booster vaccines.

All groups, except Group VI, received two primer vaccines with Ad26ₘₒₛ at weeks 0 and 12, followed by a first booster vaccine at 24 weeks. A subsequent booster vaccine was administered at 52 weeks.

In particular, Group II received two primer vaccines of Ad26ₘₒₛ, followed by two booster vaccines with 250 μg clade C Env gp140 trimeric protein (SEQ ID NO: 5) dosed with the adjuvant aluminum phosphate (hereinafter referred to as “gp140 drug product” or “gp140 DP”). Group III received two primer vaccines of Ad26ₘₒₛ, followed by two booster vaccines with co-delivered Ad26ₘₒₛ and the gp140 DP. Group IV received two primer vaccines of Ad26ₘₒₛ, followed by two booster vaccines with a composition containing two different MVA vectors, with one MVA vector expressing a mosaic Env1 gene (SEQ ID NO: 1) and a mosaic GagPol1 gene (SEQ ID NO: 3), and the other MVA vector expressing a mosaic Env2 gene (SEQ ID NO: 2) and a mosaic GagPol2 gene (SEQ ID NO: 4), with the genes being at separate locations on the vectors. The MVA vectors are termed “MVA.mos1Env/Gag-Pol” and “MVA.mos2Env/Gag-Pol,” and are collectively referred to as “MVAₘₒₛ.” Group V received two primer vaccines of Ad26ₘₒₛ, followed by two booster vaccines with co-delivered MVAₘₒₛ and the gp140 DP. The vaccine regimens tested on NHPs are summarized in Table 1A below.

TABLE 1A
Vaccine regimens tested on NHPs.

| Group | 0 weeks | 12 weeks | 24 weeks | 52 weeks |
|---|---|---|---|---|
| Group I | Ad26ₘₒₛ¹ | Ad26ₘₒₛ | Ad26ₘₒₛ | Ad26ₘₒₛ |
| Group II | Ad26ₘₒₛ | Ad26ₘₒₛ | gp140 DP³ | gp140 DP |
| Group III | Ad26ₘₒₛ | Ad26ₘₒₛ | Ad26ₘₒₛ + gp140 | Ad26ₘₒₛ + gp140 |
| | | | DP | DP |
| Group IV | Ad26ₘₒₛ | Ad26ₘₒₛ | MVAₘₒₛ² | MVAₘₒₛ |
| Group V | Ad26ₘₒₛ | Ad26ₘₒₛ | MVAₘₒₛ + gp140 | MVAₘₒₛ + gp140 |
| | | | DP | DP |
| Group VI | Sham | Sham | Sham | Sham |

¹Ad26ₘₒₛ = Ad26.mos1Gag-Pol + Ad26.mos1Env + Ad26.mos2Gag-Pol (5 × 10¹⁰ vp in total)
²MVAₘₒₛ = MVA.mos1Env/Gag-Pol + MVA.mos2Env/Gag-Pol (1 × 10⁸ pfu in total)
³gp140 DP = purified clade C Env gp140 trimeric protein dosed with an adjuvant (250 μg protein + 0.425 mg aluminum phosphate) prepared by extemporaneous mixing

The following initial core assay experiments, including ELISA binding antibody assays, antibody-dependent cellular phagocytosis (ADCP) assays, and ELISPOT assays were performed on samples taken from the NHPs treated according to the regimens described in Table 1A at 28 weeks and/or 54/56 weeks following the initial administration of the primer vaccine. A simian/human immunodeficiency virus (SHIV) challenge experiment was performed from week 72 onwards with 6 weekly applied heterologous, neutralization-resistant intra-rectal SHIV-SF162P3 challenges.

ELISA Binding Antibody (Ab) Assay

HIV-1-specific humoral response was determined at 28 and 56 weeks by a modified enzyme-linked immunosorbent assay (ELISA). The wells in one column of 96-well flat-bottomed plates (Nunc) were coated with 10 μg of clade C (C97ZA.012) gp140 coating protein (SEQ ID NO: 5), or 10 μg of mosaic 1 protein (SEQ ID NO: 6) diluted in 10 mL of 1×Dulbecco's Phosphate Buffered Saline (DPBS) (Gibco/Life Technologies) at 100 μL per well, and incubated overnight at 4° C. A known positive serum sample from an earlier study was used as a positive control, and a pre-vaccination serum sample was used as a negative control.

Plate-wells were washed once with 200 μL of ELISA Wash (1000 mL PBS (1×) and 0.5 mL Tween 20 (Sigma)). Wells were blocked with 250 μL of blocking solution (Blocker Casein in PBS (Pierce)) and incubated at room temperature for 3-4 hours. After incubation, the blocking solution was discarded. Then, 150 μL of blocking solution and 6 μL of the sample serum were added to the first column of each plate and 100 μL blocking solution in all other wells. Serial dilutions of 50 μL into 100 μL of blocking solution were then performed across the plate, and 50 μL were discarded from the final column so each well had 100 μL of sample. Plates were incubated at room temperature for 1 hour. The contents of the wells were discarded, and then the wells were washed 3 times with 200 μL of ELISA Wash.

Then, 100 μL of 1:2000 secondary antibody Peroxidase-AffiniPure Goat anti-human IgG (Jackson ImmunoResearch Labs) in blocking solution were added to each well. Plates were again incubated at room temperature for 1 hour and washed 3 times with ELISA Wash. The wells were developed with 100 μL of SeruBlue TMB Microwell Solution (KPL Laboratories), and development was stopped after 0.5 min with 100 μL of TMB Stop Solution (KPL Research Products).

The plates were read on an ELISA plate reader at 450 nm and 550 nm (Molecular Devices-Versamax, and Softmax Pro 4.7.1 software). ELISA EC₉₀ titers were calculated using the following equation (I), in which the variables were derived from the 4-parameter curve fit generated by SoftMaxPro:

$${EC}_{F} = {( \frac{F}{100 - F} )}^{VH} \cdot {EC}_{50} , \qquad ( I )$$

wherein H represents the slope and F represents the percent response.

Statistical analyses of data were performed by nonparametric comparison with control using the Dunn method for joint ranking, and the group with the highest geometric mean titer was defined as control, respectively.

Based on the results from the clade C gp140 (C97) and Mosaic 1 (Mos1) ELISA assay experiments at week 28 and week 56, Clade C gp140 Env and Mosaic 1 Env antigens displayed good correlation with no bias (data not shown).

Antibody Dependent Cellular Phagocytosis (ADCP) Assay

Functional non-neutralizing antibody responses were measured using immunoglobulin G (IgG) antibodies purified from serum samples obtained at week 28 from the treated NHPs. IgG was purified using Melon Gel columns (Thermo Scientific), and quantitated using a Nanodrop spectrophotometer (Thermo Scientific). ADCP assays were performed as described in Ackerman et al. (2011) (A robust, high-throughput assay to determine the phagocytic activity of clinical antibody samples. J. Immunol. Methods 366, 8-19), which is incorporated by reference herein in its entirety.

More specifically, clade C (C97) Env (SEQ ID NO: 5) and Mosaic M (mos 1) (SEQ ID NO: 6) Env biotinylated antigen were incubated with 1 μm yellow-green fluorescent neutravidin beads (Invitrogen) overnight. The beads were then washed and resuspended at a final dilution of 1:100 in Phosphate Buffered Saline-Bovine Serum Albumin (PBS-BSA). Antibodies purified from the serum samples and 9×10⁵ antigen-labelled beads were mixed in a round-bottom 96-well plate, and the plate was incubated for 2 hours. Human monocytic cells derived from acute myeloid leukemia (THP-1 cells; 2×10⁴ cells) were then added to each well in a final volume of 200 μL, and the plate was incubated overnight.

The next day, half the culture volume was removed and replaced with 100 μL of 4% paraformaldehyde before the plates were analyzed on a BD LSR II Flow Cytometer equipped with an HTS plate reader. For analysis, the samples were gated on live cell, and the proportion of THP-1 cells phagocytosing beads was determined. A phagocytic score was calculated as follows: (percent bead positive) multiplied by (mean fluorescence intensity bead positive).

The results obtained in the ADCP Assay at week 28 showed the phagocytic score responses of individual animals (data not shown). Statistical analyses of data were performed by nonparametric comparisons for all pairs using the Dunn method for joint ranking.

Clade C gp140 Env and Mosaic M Env antigens displayed good correlation with no bias, which are consistent with the results from the ELISA assay described above, and the neutralizing antibody (nAb) assay described below.

Neutralizing Antibody (nAb) Assay

Neutralizing antibody (nAb) responses against tier 1 HIV-1 Env pseudoviruses were measured using luciferase-based virus neutralization assays in TZM.bl cells. Specifically, viruses in the tier 1 panel included MW965.26 (clade C), SF162.LS (clade B), MN-3 (clade A), DJ263.8 (clade A), and BaL.26 (clade B).

Briefly, 96-well flat bottomed-plates were coated with serum samples obtained from the NHPs at week 56, and three-fold dilutions of the serum samples in 1004, of 10% Dulbecco's Modified Eagle Medium (DMEM) were made. Then, 200 TCID₅₀ of virus (tissue culture infectious dose, or the amount of a pathogenic agent that will produce pathological change in 50% of cell cultures inoculated) was added to each well in a volume of 50 μL. The plates were incubated for 1 hour at 37° C. TZM.bl cells were then added at 1×10⁴ cells/well in a volume of 1004, 10% DMEM containing DEAE-Dextran (Sigma) at a final concentration of 11 μg/mL.

The IC₅₀ was calculated as the serum dilution that resulted in 50% reduction in relative luminescence units as compared to undiluted virus control, after the subtraction of cell control relative luminescence units (TZM.bl cells with no virus present).

The results from the nAb assay, i.e., the HIV-1 tier 1 TZM-bl neutralization assays against MW965.26 (clade C), SF162.LS (clade B), MN-3 (clade A), DJ263.8 (clade A), and BaL.26 (clade B) in samples obtained from the NHPs at week 56 (data not shown) are consistent with the results from the ELISA assay.

ELISPOT Assay

HIV-1-specific cellular immune responses were assessed by IFN-γ ELISPOT assays as previously described in Liu et al., 2009, Nature 457: 87-91, which is herein incorporated by reference in its entirety. ELISPOT assays utilized pools of HIV-1 potential T-cell epitope (PTE) peptides covering global potential human T cell epitopes. In earlier studies, analyses of cellular immune breadth utilized subpools of 10-16 peptides covering each antigen followed by epitope mapping using individual peptides, essentially as we have previously reported in Barouch et al., 2010, Nat. Med. 16:319-323 [54], which is incorporated by reference herein in its entirety. Epitope-specific CD8+ and CD4+T lymphocyte responses were determined by cell depletion studies.

Briefly, immunogenicity of the treated NHPs was assessed in samples obtained at week 54 by IFN-γ ELISPOT assays using PTE peptide pools. Peripheral blood mononuclear cells (PBMCs) were stimulated with the PTE peptide pools, and after incubation, the cells were washed, labeled, and developed to visualize spot forming cells.

Study Conclusions

Results from this preclinical animal study indicated that a combination of rAd26 vectors and/or MVA vectors with isolated antigenic polypeptide in prime-boost combinations is useful for raising broad HIV-specific humoral and cellular immune responses in primates. Specifically, the utility of incorporating a gp140 protein in one or more boosting immunizations in raising broad HIV-specific humoral and cellular immune responses in primates was demonstrated. Moreover, all vaccine regimens tested were shown to be immunogenic in all immunized animals (Group II-V).

In particular, the administration of one or more rAd26 vectors (week 0 and 12) expressing one or more HIV-1 antigens followed by a boosting immunization at weeks 24 and 52 with rAd26 vectors or MVA vectors and an isolated clade C gp140 protein, resulted in efficient boosting of the humoral response to HIV-1, as shown by the results of the ELISA and ADCP assays (data not shown). Furthermore, administration of one or more rAd26 vectors, followed by a boosting immunization at weeks 24 and 52 with MVA vectors with or without clade C gp140 protein was able to significantly increase cellular immune responses as measured by ELISPOT assay (data not shown).

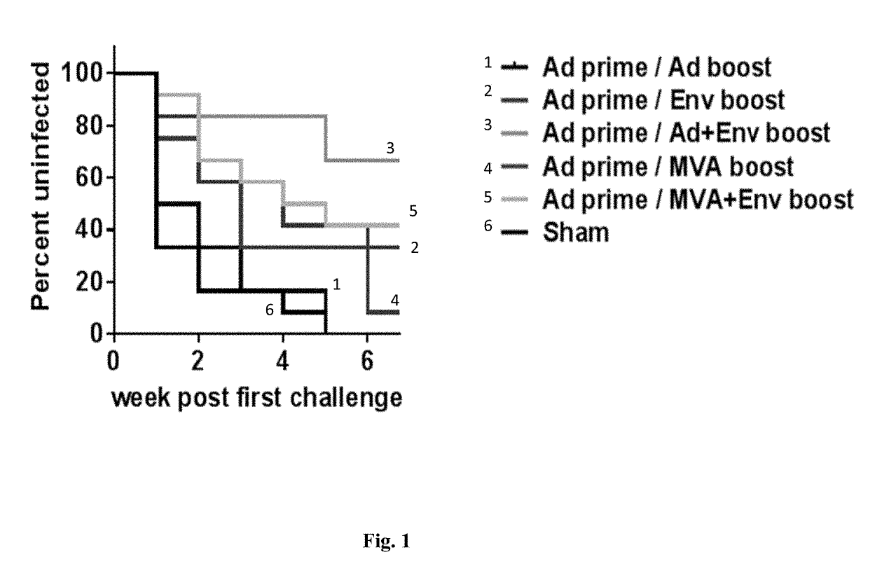

Results from the SHIV challenge experiment are shown in FIG. 1 and Table 2B. The reported data reflects only the envelope antigen component of the vaccine (e.g., HIV antigenic polypeptides having the amino acid sequences of SEQ ID NOs: 1 and 2), since there is no significant cross-reactivity between HIV Gag/Pol antigens (e.g., HIV antigenic polypeptides having the amino acid sequences of SEQ ID NOs: 3 and 4) versus SIV Gag/Pol antigens.

TABLE 2B
Results of SHIV Challenge in NHPs

| | Per-Exposure Risk | Full Protection After |
|---|---|---|
| Group No. | Reduction | 6 Challenges |
| 1. (rAd26/rAd26) | 35% | 0% |
| 2. (rAd26/Env) | 84% | 33% |
| 3. (rAd26/rAd26 + Env) | 94% | 66% |
| 4. (rAd26/MVA) | 71% | 8% |
| 5. (rAd26/MVA + Env) | 87% | 42% |
| 6. (Sham) | N/A | 0% |

As demonstrated by the results of the study, Group (regimen) 3, in which NHPs were treated with rAd26 prime followed by rAd26+HIV gp140 protein boost provided significantly better protection against SHIV challenge than any of the other regimens tested. In particular, vaccination according to regimen 3 had reduced the per-exposure risk of SHIV infection in the NHPs by 94 percent and resulted in 66 percent complete protection after six exposures.

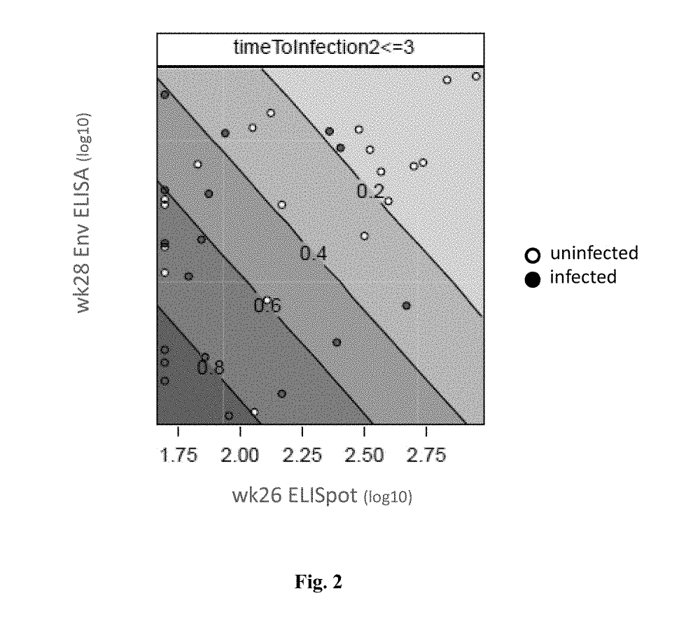

As shown in FIG. 2, binding antibodies to HIV Env (x-axis, ELISA responses) together with HIV Env specific T cells (y-axis, ELISpot responses) correlate with protection in NHP SHIV_{SF162P3} challenge study. In addition, functional antibodies as assessed by ADCP were also found to correlate with protection NHP SHIV_{SF162P3} challenge study, as has been observed in previous studies.

### Example 2. Study of HIV Vaccine Regimens in Humans

The following multicenter, randomized, parallel group, placebo-controlled, double-blind clinical study in healthy HIV-uninfected adult men and women was performed: A Phase 1/2a Study to Evaluate the Safety/Tolerability and Immunogenicity of Homologous Ad26 Mosaic Vector Vaccine Regimens or Ad26 Mosaic and MVA Mosaic Heterologous Vector Vaccine Regimens, with High-Dose, Low-Dose or no Clade C gp140 Protein Plus Adjuvant for HIV Prevention. Data for this study are shown herein.

Overall Rationale

A study was performed to assess the safety/tolerability and immunogenicity of seven prime-boost vaccine regimens. Subjects received four doses of study vaccine: Ad26ₘₒₛ or placebo was given at weeks 0 and 12; and Ad26ₘₒₛ or MVAₘₒₛ, both with or without glycoprotein 140 drug product (low or high dose), or placebo only was given at Weeks 24 and 48.

Study vaccines used were Ad26ₘₒₛ, MVAₘₒₛ and gp140 DP as follows (see also Example 1):

(i) Ad26ₘₒₛ was composed of the following three vaccine products supplied in the same vial and administered in a 2:1:1 ratio: Ad26.Mos1Env, Ad26.Mos1Gag-Pol, and Ad26.Mos2Gag-Pol expressing HIV-1 mosaic Env1 (SEQ ID NO: 1), mosaic GagPol1 (SEQ ID: NO 3), and mosaic GagPol2 (SEQ ID NO: 4) genes, respectively;
(ii) MVAₘₒₛ was composed of the following two vaccine products supplied in separate vials and administered in a 1:1 ratio: MVA-Mosaic1 (MVA virus expressing Mosaic1 HIV-1 Gag, Pol, and Env proteins having SEQ ID NOs: 1 and 3) and MVA-Mosaic2 (MVA virus expressing Mosaic2 HIV-1 Gag, Pol, and Env proteins having SEQ ID NOs: 2 and 4); and
(iii) gp140 drug product contained HIV-1 Clade C glycoprotein 140 (recombinant trimeric gp140 having SEQ ID NO: 5), produced by a transformed PER.C6® cell line constructed to produce gp140. In this study, gp140 drug product was dosed with aluminum phosphate as adjuvant, and the dosed gp140 drug product is simply referred to as “gp140 DP.”

Objectives

The primary objectives of the study included (1) assessing the safety/tolerability of various prime-boost regimens containing Ad26ₘₒₛ, MVAₘₒₛ, and/or gp140 DP components; and (2) comparing HIV Env binding antibody responses between the different vaccine regimens.

The secondary objective of the study included assessing other antibody binding, antibody effector function and antibody characterization, and cellular responses.

The exploratory objectives of the study include (1) exploring immune responses to the different vaccine regimens in mucosal secretions in a subset of subjects; (2) exploring gene expression patterns between the different vaccine regimens; and (3) exploring neutralization antibodies against the Ad26 vectors.

Vaccination and Experimental Design

The study comprised of a 48-week vaccination period during which subjects were vaccinated at baseline (Week 0), Week 12 and Week 24, with a booster at Week 48, and a 48-week follow-up period to a final visit at Week 96. Vaccinations were administered as shown in Table 1B, and blood samples were taken at specific clinic visits to assess immune responses.

A long-term follow-up period (approximately 2 years after Week 96) will continue for subjects randomized to the regimen that are subsequently selected for future studies, based on the analysis of the Week 28 data. If the Week 28 data were inconclusive, then Week 52 data were taken into consideration in regimen selection. In the event that no clear decision can be made, this extended follow-up period can include subjects from more than one group with the purpose of assessing durability of immune responses. The end of the study is the last subject's final visit.

TABLE 1B
Vaccine regimens tested on humans

| Group | N | Week 0 (baseline) | Week 12 | Week 24 | Week 48 booster |
|---|---|---|---|---|---|
| Group 1 | 50 | Ad26ₘₒₛ | Ad26ₘₒₛ | Ad26ₘₒₛ + gp140 DP | Ad26ₘₒₛ + gp140 DP |
| | | | | (250 μg protein/adj*) | (250 μg protein/adj*) |
| Group 2 | 50 | Ad26ₘₒₛ | Ad26ₘₒₛ | Ad26ₘₒₛ + gp140 DP | Ad26ₘₒₛ + gp140 DP |
| | | | | (50 μg protein/adj*) | (50 μg protein/adj*) |
| Group 3 | 50 | Ad26ₘₒₛ | Ad26ₘₒₛ | Ad26ₘₒₛ | Ad26ₘₒₛ |
| Group 4 | 50 | Ad26ₘₒₛ | Ad26ₘₒₛ | MVAₘₒₛ + gp140 DP | MVAₘₒₛ + gp140 DP |
| | | | | (250 μg protein/adj*) | (250 μg protein/adj*) |
| Group 5 | 50 | Ad26ₘₒₛ | Ad26ₘₒₛ | MVAₘₒₛ + gp140 DP | MVAₘₒₛ + gp140 DP |
| | | | | (50 μg protein/adj*) | (50 μg protein/adj*) |
| Group 6 | 50 | Ad26ₘₒₛ | Ad26ₘₒₛ | MVAₘₒₛ | MVAₘₒₛ |
| Group 7 | 50 | Ad26ₘₒₛ | Ad26ₘₒₛ | gp140 DP | gp140 DP |
| | | | | (250 μg protein/adj*) | (250 μg protein/adj*) |
| Group 8 | 50 | Placebo | Placebo | Placebo | Placebo |

*adj is AdjuPhos ® (sterilized aluminum phosphate wet gel suspension; used as adjuvant for gp140; aluminum content is 0.425 mg/0.5 mL dose; 50 μg (low dose) and 250 μg (high dose) refer to total protein content of gp140 protein.

Dosage and Administration

Subjects received doses of study vaccine at four time points according to randomization, on Day 1 of Week 0, at Week 12, and at Week 24, with a booster at Week 48, administered by intramuscular injection into the deltoid. For visits with only one injection (i.e., at Weeks 0 and 12), either deltoid could be used for the injection. When two study vaccine injections were given at one visit (i.e., at Weeks 24 and 48), a different deltoid was used for each injection (with exceptions allowed upon medical indication). Study vaccines with the administered doses are as follows:

### (i) Ad26ₘₒₛ, (Ad26.Mos1Env+Ad26.Mos1 Gag-Pol+Ad26.Mos2Gag-Pol):

Total dose was 5×10¹⁰ viral particles (vp) per 0.5 mL injection

### (ii) MVAₘₒₛ (MVA-Mosaic1+MVA-Mosaic2):

Total dose was 10⁸ plaque-forming units (pfu) per 0.5 mL injection
(iii) gp140 DP:
Low-dose (gp140 LD): gp140 DP with 50 μg total protein, mixed with aluminum phosphate adjuvant (0.425 mg aluminum) at the pharmacy, per 0.5 mL injection
High-dose (gp140 HD): gp140 DP with 250 μg total protein, mixed with aluminum phosphate adjuvant (0.425 mg aluminum) at the pharmacy, per 0.5 mL injection

### (iv) Placebo:

0.9% saline, 0.5 mL injection

Immunogenicity Evaluations

Assays were performed to evaluate humoral immune responses including, but not limited to: Env-specific serum binding antibody assay, nAb assays, and antibody-dependent cellular phagocytosis (ADCP) assay, as well as epitope mapping (see Table 2).

TABLE 2
Humoral Immune Response Assays

| Objective/ | | | | |
|---|---|---|---|---|
| endpoint | System | Assay/Method | Readout | Timepoint |
| Primary | Serum | Env binding antibody | Titer or % responders | Baseline |
| | | (ELISA) | (Clade C) | 1 mo post-vac. 1 |
| | | | and breadth | 0.5, 1 mo post-vac. 2-4 |
| | | | (Clade A, B, C) | 3, 6 mo post-vac. 4 |
| Secondary | Serum | HIV neutralizing | Tier 1 and Tier 2^{a} nAbs: | As above |
| | | antibody | GMT for each isolate, | |
| | | | % responders to each isolate | |
| | | | Breadth: # isolates neutralized | |
| Secondary | Serum | gp120 binding | Anti-gp120 titer | As above |
| | | antibody | (Clade A, B, C) | |
| Secondary | Scrum | ADCP | % phagocytosis | As above |
| Secondary | Serum | Isotyping Env binding | Isotyping (Clade C) | As above |
| | | antibody (ELISA) | (IgA, IgG1, IgG2, IgG3) | |
| Exploratory | Serum | Epitope mapping | Targeted epitopes and diversity | 1 mo post-vac. 1-4 |
| | | | (including V2) | At vac. 2-4 |
| Exploratory | Serum | Ad26 neutralization | Titers of Ad26 neutralization | 1 mo post-vac. 1-4 |
| | | antibodies | antibodies | 3, 7.5, 12 mo post-vac. 4 |
| | | | | At vac. 1-4 |

ADCP = antibody-dependent cellular phagocytosis;
ELISA = enzyme-linked immunosorbent assay;
GMT = geometric mean titer;
Ig = immunoglobulin;
mo = month;
nAb = neutralizing antibody;
vac = vaccination
^{a}Classification of HIV-1 viruses according to sensitivity to antibody-mediated neutralization: very high (tier 1A), above-average (tier 1B), moderate (tier 2), or low (tier 3)¹. Tier 2 will only be assessed if Tier 1 shows positive results

Assays were performed to evaluate cellular immune responses including, but not limited to: ELISPOT, intra-cellular cytokine staining, and multi-parameter flow cytometry (see Table 3).

TABLE 1
T-Cell Immune Response Assays

| Objective/ | | | | |
|---|---|---|---|---|
| endpoint | System | Assay/Method | Readout | Timepoint |
| Secondary | PBMC | ELISPOT | Breadth and depth: | Baseline 0.5, |
| | | | # peptides, % responders, | 1 mo post-vac. 3 & 4 |
| | | | median response | |
| Exploratory | PBMC | Intracellular | % of CD4 and CD8+ T cells | Baseline |
| | | cytokine staining | producing IFNγ, IL-2, TNFα | 1 mo post-vac. 1 |
| | | | | 0.5, 1 mo post-vac. 2-4 |
| | | | | 3, 7.5 mo post-vac. 4 |
| Exploratory | PBMC | Multi-parameter | Characterization of memory T-cell | Baseline |
| | | flow cytometry | development with emphasis on | 1 mo post-vac. 1 |
| | | | follicular helper T cells | 0.5, 1 mo post-vac. 2-4 |
| | | | | 3, 7.5 mo post-vac. 4 |
| Exploratory | PBMC | Gene expression | Regulation of genes (clusters) that | Baseline |
| | | analysis | predict specific immune responses | 1 mo post-vac. 1 |
| | | | and HLA typing | 0.5, 1 mo post-vac. 2-4 |
| | | | | 3, 7.5 mo post-vac. 4 |

ELISPOT = enzyme-linked immunospot assay;
HLA = human leukocyte antigen;
INFγ = interferon gamma;
IL-2 = interleukin 2;
mo = month;
PBMC = peripheral blood mononuclear cell;
TNFα = tumor necrosis factor alpha;
vac = vaccination
Note:
HLA only tested once (using the baseline blood sample)

Safety Results

The study is currently ongoing and is still blinded for subjects and sites. At the time of this analysis, when all subjects received 4^{th} vaccination or discontinued earlier, 393 subjects were randomized and received at least one dose of study vaccine or placebo.

Most solicited adverse events (AEs) were grade 1 and 2, and among which, most common were injection site pain, headache, fatigue. There were no clear differences in the solicited AEs between groups that participated in the study. Most unsolicited AEs were grade 1 and 2 and were unrelated. There were no clear differences in the unsolicited AEs between groups that participated in the study. There was only 1 related serious AE, hypersensitivity with multiple confounding factors.

Thus, all of the vaccine regimens were safe and generally well-tolerated by participants.

Immunogenicity Results

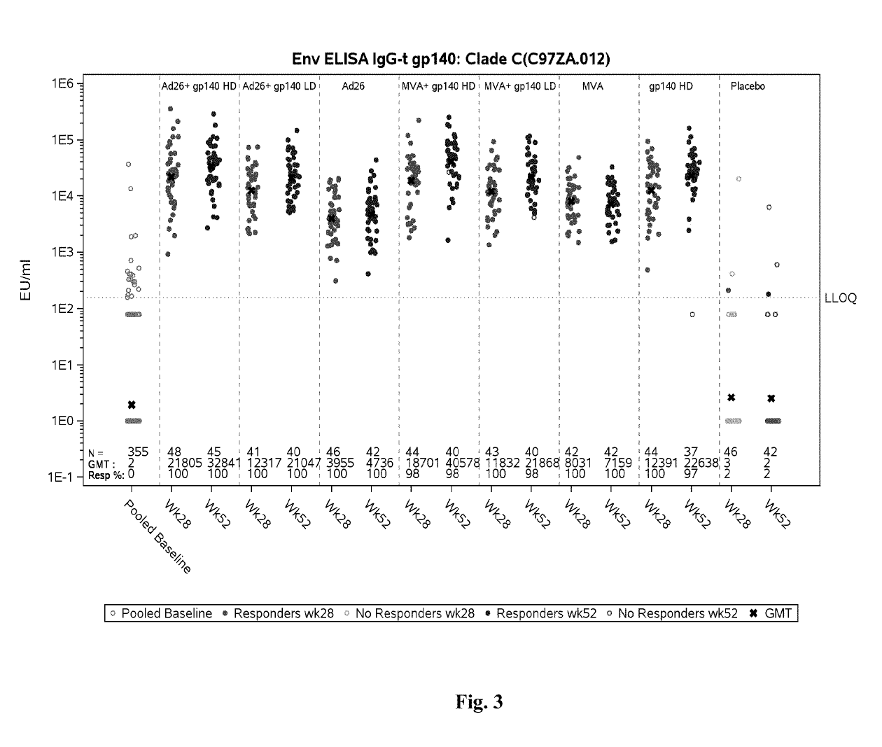

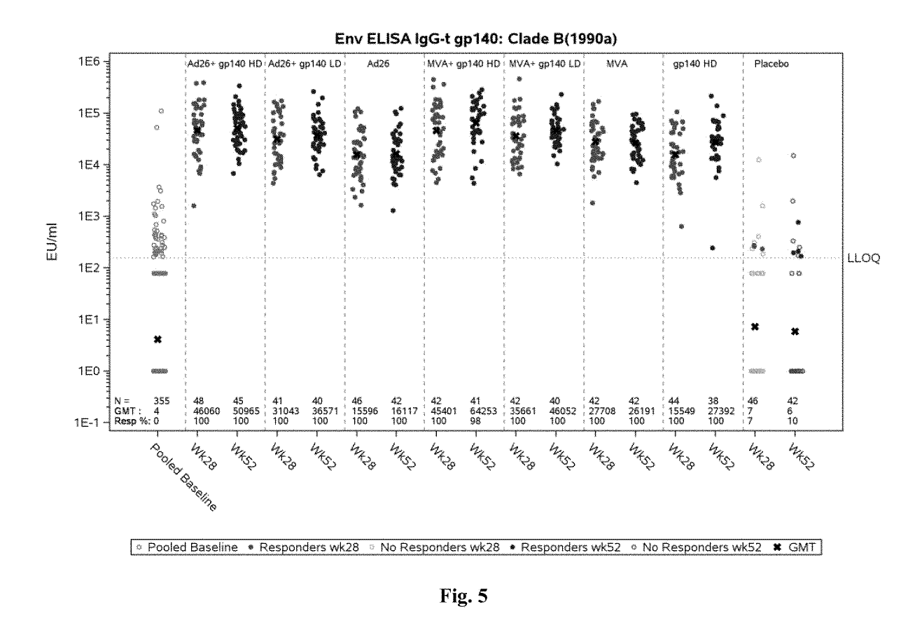

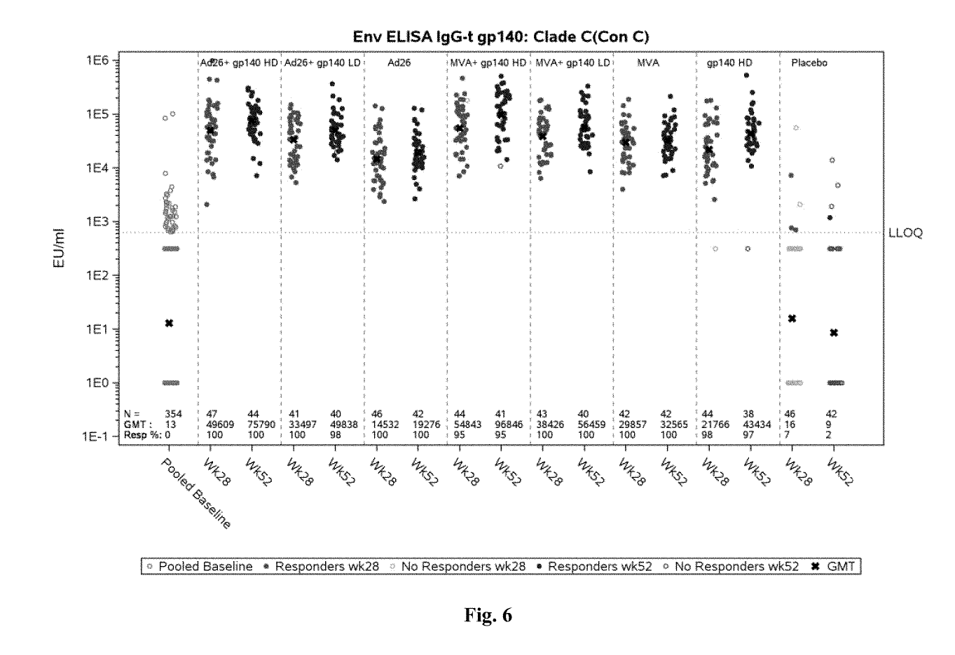

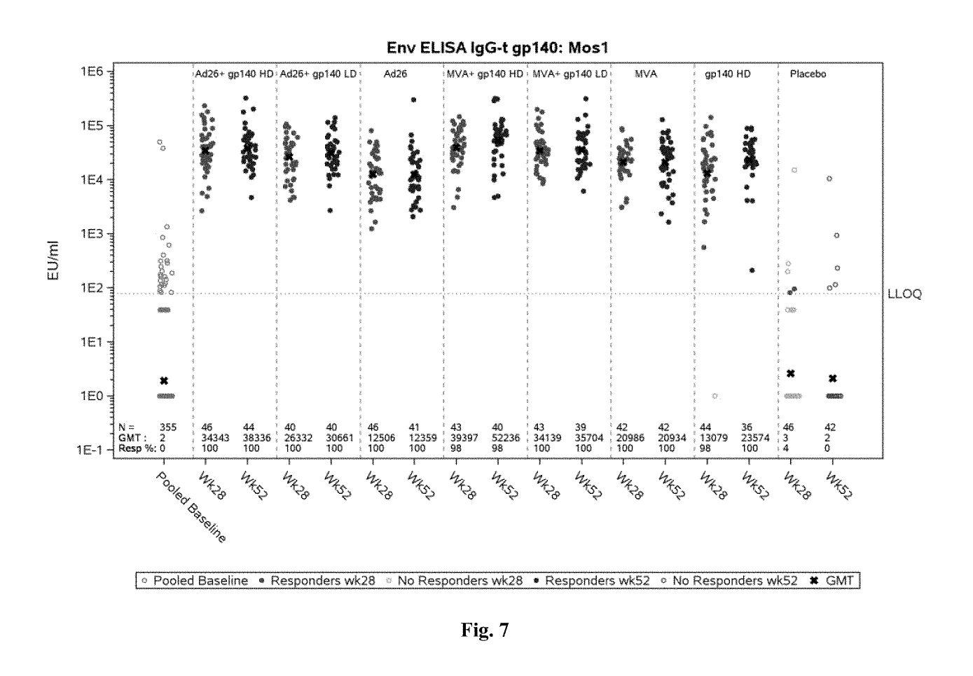

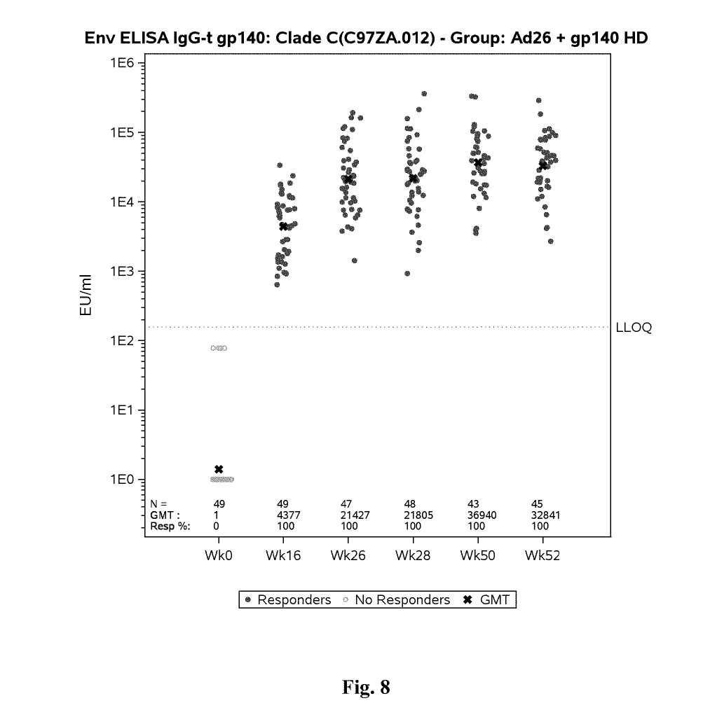

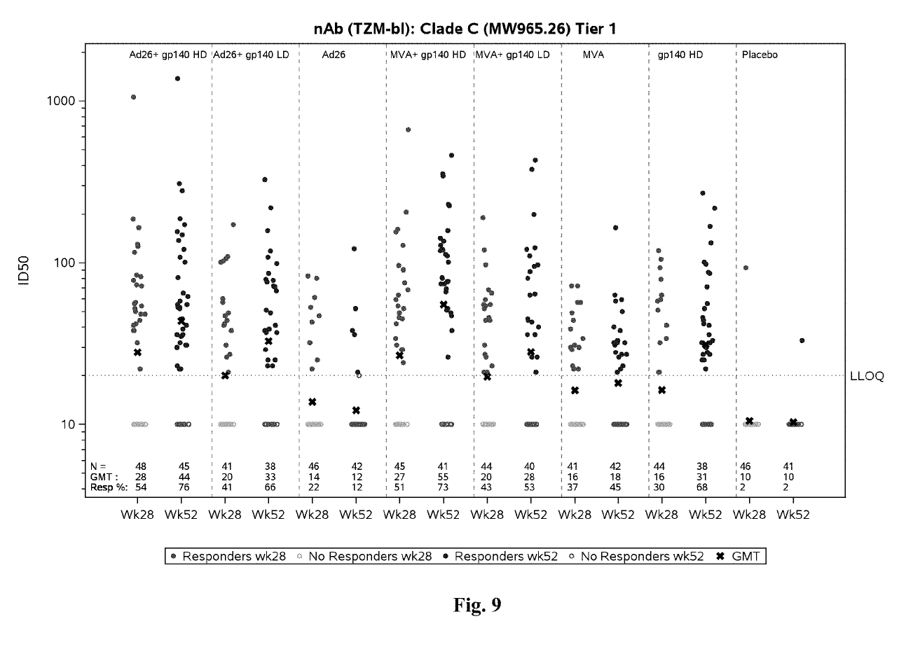

Results are shown in FIGS. 3-14. All active vaccine regimens were immunogenic.

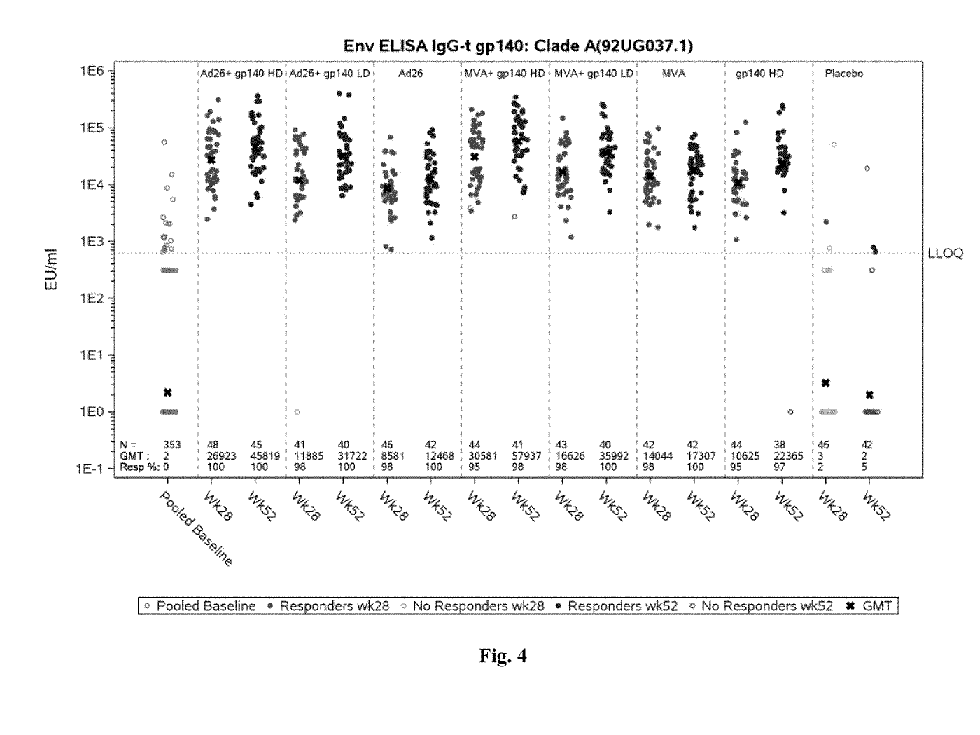

Relating to the humoral immunogenicity, it was observed that: there was about 100% responders in most groups after the third vaccination; there was a clear contribution of gp140 in the boost, and of the gp140 dose; there was a detectable contribution of the vector to the gp140 boost, which seemed more evident in the Ad26 boosted groups than in the MVA boosted groups. The number of responders after the fourth vaccination was maintained and a slight increase in ELISA titers was observed in most groups that included gp140 in the boost (FIGS. 3-8); cross-clade responses (e.g., against gp140 of clades A, B, consensus C, mosaic 1) were detected with very similar response patterns as to the vaccine clade C (FIGS. 4-7); groups with vectors+gp140 HD (250 μg) rank best for overall humoral responses (FIGS. 3-10); kinetics of binding antibodies show 100% responders after 2 administrations of Ad26ₘₒₛ, with boosts in titer after protein (FIG. 3-8).

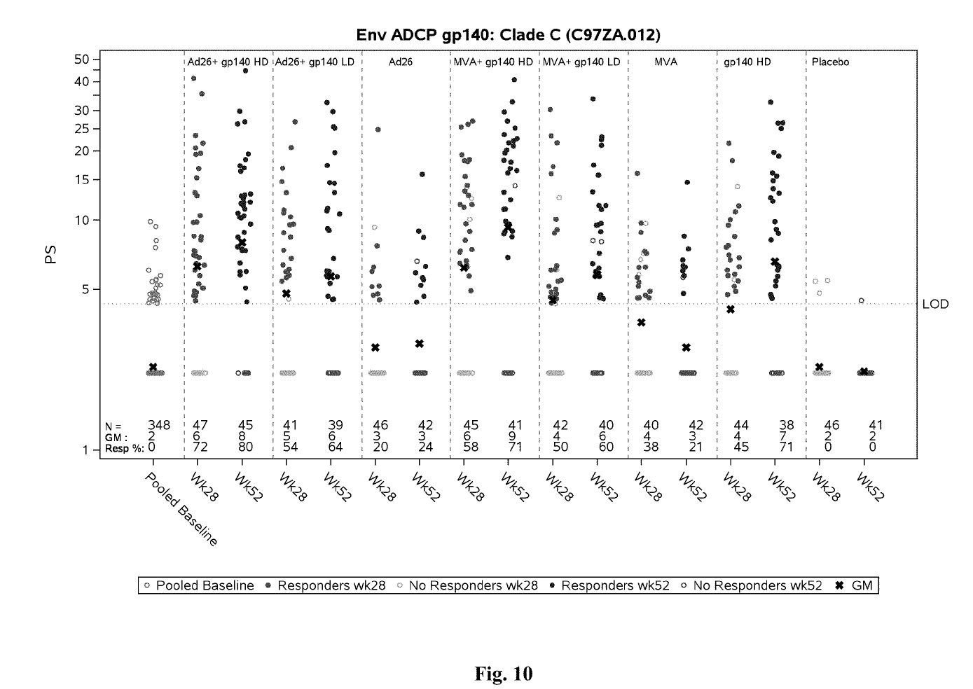

All vaccine containing regimens elicit ADCP responses, with a clear contribution of gp140 boost and dose; after the second boost the number of responders were maintained with slight increase in ADCP titers in protein boosted groups (FIG. 10). After the first boost (week 28 data), the regimen with Ad26 and gp140 HD in the boost showed 72% responders against clade C gp140 (FIG. 10), and 36% responders against clade A gp140, 79% responders against clade B gp140, over 90% responders against mosaic gp140 (data not shown). Data without protein boost showed only 37% responders for ADCP against clade B gp140, for example (not shown).

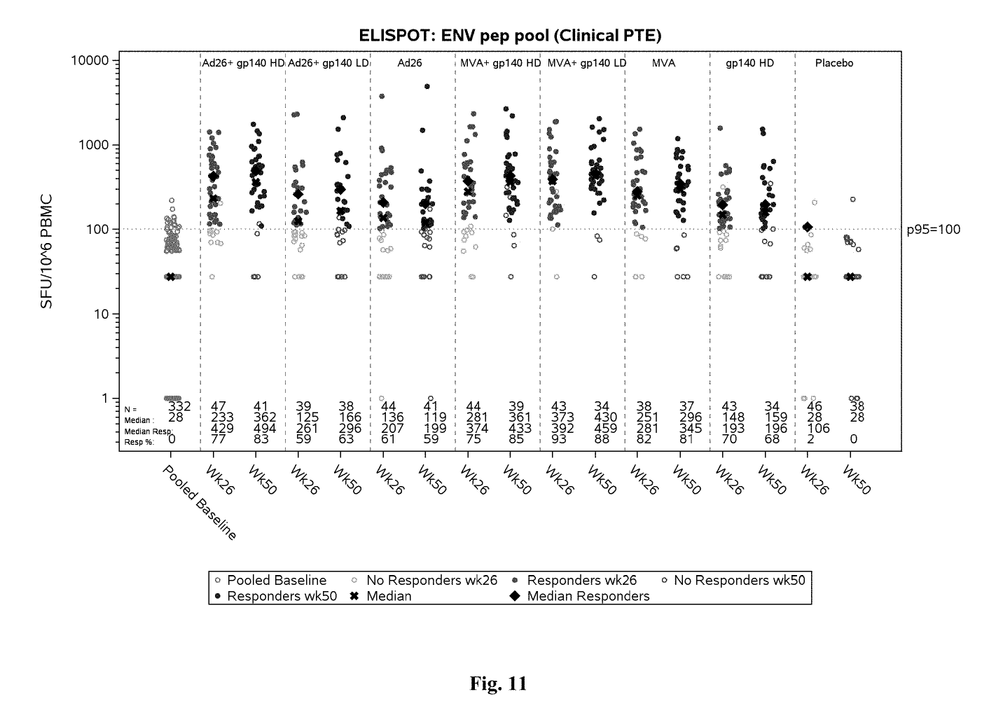

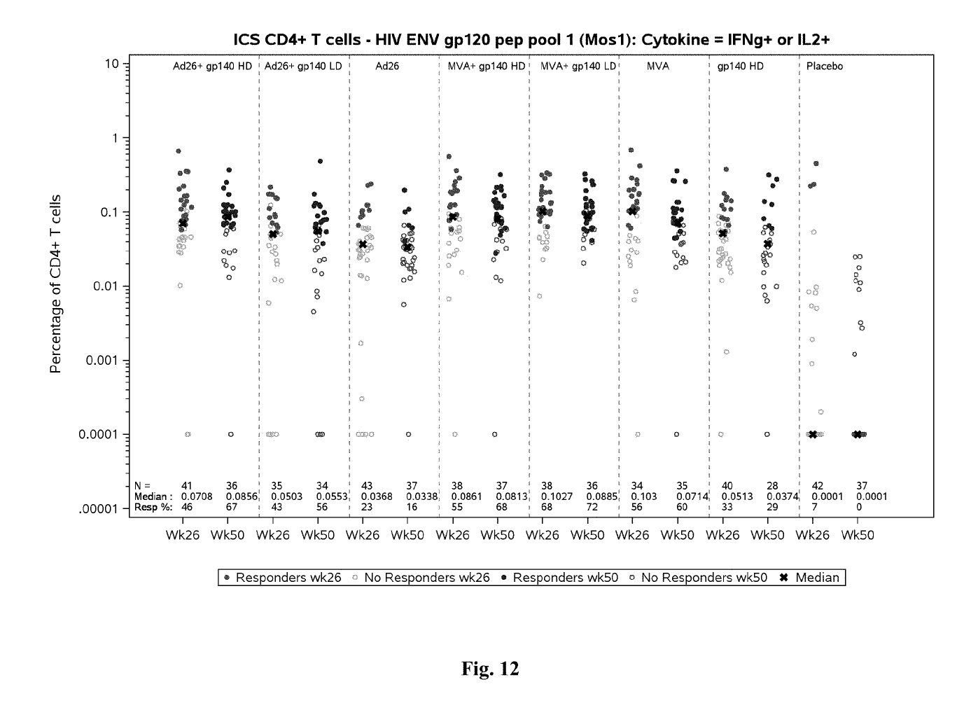

With regard to the cellular immunogenicity (ELISPOT responses to the potential T-cell epitopes (PTE) peptide pool), a high percentage of responders was shown in most groups (FIG. 11), again confirming that all vaccines were immunogenic. The highest cellular immunogenicity was observed in the Ad26+gp140 HD and in the MVA+gp140 boosted groups. After the second boost (4^{th} administration) the cellular responses were maintained or slightly increased. Also observed were a high frequency of responders and sustained magnitude of responses in Gag and Pol ELISPOT (not shown); and slightly increased number of responders for Env specific CD4 T cells producing IFNγ+/−IL-2, in groups boosted with vector and protein (FIG. 12).

More specifically, to test the vaccine take after priming, the group of individuals who comprised the Ad26/Ad26+gp140HD regimen (Group 1) were also tested for Clade C gp140 response by ELISA after two prime vaccinations and 100% of participants had a detectable humoral response. Post the third vaccination, most groups showed 100% of participants having a detectable antibody response against clades B and C and these response rates were maintained through the fourth vaccination across all regimens tested (FIGS. 3&5). The ELISA utilizing the same ELISA antigen as used for protein boost immunization showed a clear increase in binding antibody titers in cohorts receiving a Clade C gp140 boost (irrespective of vector boost). About 100% antibody response rates to diverse clades A, B, C, Consensus C and Mosaic Env gp140 proteins were observed in most vaccine groups (FIGS. 3-7). The magnitude of ELISA response across antigens increased after the fourth vaccination in groups boosted with gp140. The magnitude of ELISA responses showed a clear increase in antibodies upon boost containing a high dose (250 μg) of the Clade C gp140 compared to the low dose (50 μg), and increase with inclusion of either vector (Ad26 or MVA) in the boost along with the Clade C gp140 in comparison to Clade C gp140 alone for HIV gp140 Env antigens. This pattern of humoral responses was similar across HIV Env Clades A, B, C as well as Consensus Clade C and Mosaic constructs. In addition, for the autologous HIV Env Clade C C97ZA.012, the geometric mean ELISA titer ratio of the Ad26/Ad26+gp140HD group versus the Ad26/Ad26 group was 5.5 after the third vaccination (with a 95% CI above 3.5) and was 6.9 after the fourth vaccination (with a 95% CI above 4.5).

Clear Env-specific IgG3, antibody dependent cellular phagocytosis (ADCP) and tier 1A neutralizing antibody (NAb) responses were detected in most groups and increased after the fourth vaccination. Inclusion of gp140 in the boost enhanced IgG3, ADCP and neutralizing antibody (Nab) generation. Similar to the total IgG response, the functional antibody response as assessed by ADCP showed a clear increase in antibodies upon boost containing a high dose of the Clade C gp140 compared to the low dose, and increase with inclusion of either vector in the boost along with the Clade C gp140 in comparison to Clade C gp140 alone (FIG. 10). IgG subclass responses to the vaccine protein were of the IgG1 and IgG3 subtype, with little to no IgG2 and IgG4 detected in most vaccine groups at any time point examined. The Ad26/Ad26+gp140HD and Ad26/MVA+gp140HD groups showed the highest humoral responses overall.

High frequencies of cellular responses were identified by ELISPOT after stimulation by Env, Gag or Pol peptide pools, either matched to the vaccine encoded inserts or generated from diverse circulating strains of HIV, with some of the highest numbers of responders in the groups boosted with Ad26+gp140HD as well as those groups with MVA in the boost (FIG. 11). Following the fourth vaccination, the magnitude of ELISpot responses to Env peptide pools increased in most groups. The highest median responses were elicited in groups with MVA in the boost. Similar response frequencies were also seen for ELISPOT measurements using Gag and Pol peptide pools, although the magnitude of responses did not change in concerted fashion after a fourth vaccination. Responses from CD4 and CD8 T cells capable of producing IFNγ and/or IL2 were detected by Intracellular Cytokine Staining (ICS). The predominant CD4 T cell responses were directed against Env, while the predominant CD8 T cell responses were directed against reverse transcriptase and Pol. The magnitude of ICS responses was conserved between three and four vaccinations, while there was an increase in the response rate of CD4 T cells responding to vector matched (Mosaic) Env peptides (FIG. 12). A clear contribution of both the vector and the protein to boosting of both humoral and cellular immune responses to Env was observed.

Results from this clinical study thus far indicate that the Ad26/Ad26+gp140HD regimen (Group 1) that showed greatest protection in monkey studies also elicited the greatest immune responses in humans among the tested regimens. As summarized in Table 4, these results support further evaluation of the regimen comprising two primes with Ad26 vectors expressing at least three mosaic HIV antigens having the amino acid sequences of SEQ ID NOs: 1, 3 and 4, and two boosts with the Ad26 vectors expressing the same at least three mosaic HIV antigens and at least one isolated clade C gp140 antigen (having SEQ ID NO: 5) with aluminum adjuvant, preferably at high dose of antigen (about 250 μg gp140 antigen). For example, the target median response rate for ADCP responses to Clade C Env is ≥56% with the lower limit of 95% confidence interval (LL of 95% CI) of ≥40%. The Ad26/Ad26+gp140HD regimen (Group 1) resulted in ADCP responses to Clade C Env at a median response rate of at least 72% with LL of 95% CI of >57% 4 weeks post the 3^{rd} administration (week 28), and a median response rate of at least 80% with LL of 95% CI of ≥65% 4 weeks post the 4^{th} administration (week 52) (FIGS. 3-12 and Table 4).

TABLE 4
criteria towards phase 2b/proof of concept efficacy study, and results obtained in a human population

| | | Target | | Results post | Results post |
|---|---|---|---|---|---|
| Criteria | Endpoint | (LL of 95% CI) | | 3rd | 4th |
| Humoral | IgG binding responses on clade C Env | ≤90% | | 100% | 100% |
| | | (≤77%) | | (93%) | (92%) |
| | ADCP responses to Clade C Env | ≤56% | | 72% | 80% |
| | | (≤40%) | | (57%) | (65%) |
| Cellular | Elispot responses to at least one ENV | ≤50% | | 77% | 83% |
| | peptide pool* | (≤35%) | | (62%) | (68%) |
| Env boost | IgG to clade C Env of | ≤1.5 fold | | 5.5 fold | 6.9 fold |
| | Ad/Ad + Env over Ad/Ad | | | (3.5) | (4.5) |
| Magnitude | >2.15 log10 ePTE Env ELISPOT | post | post | 94% | 93% |
| | OR >3.8 log10 Clade C gp140 ELISA | 3rd: ≤60% | 4th: ≤75% | | |
| | Subjects should be above BOTH | post | post | 64% | 80% |
| | response thresholds | 3rd: ≤40% | 4th: ≤60% | | |

While ADCP, magnitude and Env boost criteria were considered supportive for decision making, the ELISA and ELISPOT criteria were considered essential (had to be met).

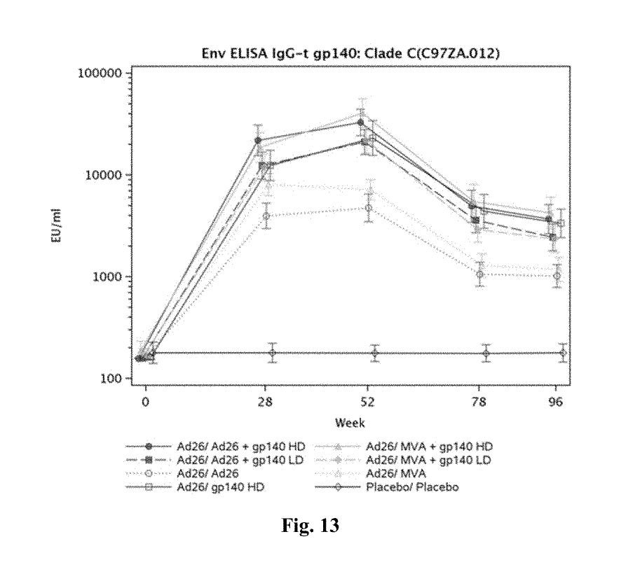

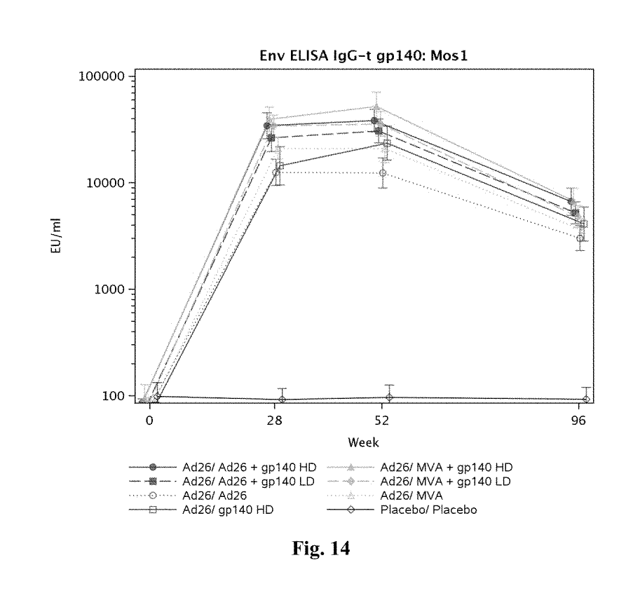

Persistent humoral and cellular immune responses were observed for 48 weeks following the final vaccination. 100% of participants in the group boosted with Ad26+gp140HD and 97% of participants in the group boosted with Ad26+gp140LD maintained humoral responses to Clade C gp140 until week 96 (FIG. 13). 100% of the participants in the groups boosted with either Ad26+gp140HD or Ad26+gp140LD maintained humoral responses to mosaic gp140 with magnitudes that indicate sustained generation of vaccine induced antibodies (FIG. 14). Similar antibody decay rates were seen irrespective of the immunogens used in the boost vaccinations. Differences in the decay rates between different gp140 antigens indicate that multiple antibody specificities were induced by vaccination and that their persistence was antigen-dependent. Env-specific cellular responses, assessed by γIFN ELISPOT responses to PTE Env peptides, showed a similar result (data not shown). This outcome was not predicted, and stands in contrast to the results seen in other HIV vaccine trials, where immune responses waned rapidly [9].

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

### REFERENCES

1. Gurwith, M., et al., Safety and immunogenicity of an oral, replicating adenovirus serotype 4 vector vaccine for H5N1 influenza: a randomised, double-blind, placebo-controlled, phase 1 study. Lancet Infect Dis, 2013. 13(3): p. 238-50.
2. Centers for Disease, Control, and Prevention, Vital signs: HIV prevention through care and treatment—United States. MMWR Morb Mortal Wkly Rep, 2011. 60(47): p. 1618-23.
3. Centlivre, M., et al., In HIV-1 pathogenesis the die is cast during primary infection. AIDS, 2007. 21(1): p. 1-11.
4. Wawer, M. J., et al., Rates of HIV-1 transmission per coital act, by stage of HIV-1 infection, in Rakai, Uganda. J Infect Dis, 2005. 191(9): p. 1403-9.
5. Flynn, N. M., et al., Placebo-controlled phase 3 trial of a recombinant glycoprotein 120 vaccine to prevent HIV-1 infection. J Infect Dis, 2005. 191(5): p. 654-65.
6. Pitisuttithum, P., et al., Randomized, double-blind, placebo-controlled efficacy trial of a bivalent recombinant glycoprotein 120 HIV-1 vaccine among injection drug users in Bangkok, Thailand. J Infect Dis, 2006. 194(12): p. 1661-71.
7. Gray, G. E., et al., Safety and efficacy of the HVTN 503/Phambili study of a clade-B-based HIV-1 vaccine in South Africa: a double-blind, randomised, placebo-controlled test-of-concept phase 2b study. Lancet Infect Dis, 2011. 11(7): p. 507-15.
8. Buchbinder, S. P., et al., Efficacy assessment of a cell-mediated immunity HIV-1 vaccine (the Step Study): a double-blind, randomised, placebo-controlled, test-of-concept trial. Lancet, 2008. 372(9653): p. 1881-93.
9. Rerks-Ngarm, S., et al., Vaccination with ALVAC and AIDSVAX to prevent HIV-1 infection in Thailand. N Engl J Med, 2009. 361(23): p. 2209-20.
10. McElrath, M. J., et al., HIV-1 vaccine-induced immunity in the test-of-concept Step Study: a case-cohort analysis. Lancet, 2008. 372(9653): p. 1894-905.
11. Abbink, P., et al., Comparative seroprevalence and immunogenicity of six rare serotype recombinant adenovirus vaccine vectors from subgroups B and D. J Virol, 2007. 81(9): p. 4654-63.
12. Vogels, R., et al., Replication-deficient human adenovirus type 35 vectors for gene transfer and vaccination: efficient human cell infection and bypass of preexisting adenovirus immunity. J Virol, 2003. 77(15): p. 8263-71.
13. Farina, S. F., et al., Replication-defective vector based on a chimpanzee adenovirus. J Virol, 2001. 75(23): p. 11603-13.
14. Barouch, D. H., et al., International seroepidemiology of adenovirus serotypes 5, 26, 35, and 48 in pediatric and adult populations. Vaccine, 2011. 29: p. 5203-5209.
15. Mast, T. C., et al., International epidemiology of human pre-existing adenovirus (Ad) type-5, type-6, type-26 and type-36 neutralizing antibodies: correlates of high Ad5 titers and implications for potential HIV vaccine trials. Vaccine, 2010. 28(4): p. 950-7.
16. Chen, H., et al., Adenovirus-based vaccines: comparison of vectors from three species of adenoviridae. J Virol, 2010. 84(20): p. 10522-32.
17. Thorner, A. R., et al., Age dependence of adenovirus-specific neutralizing antibody titers in individuals from sub-Saharan Africa. J Clin Microbiol, 2006. 44(10): p. 3781-3.
18. Sprangers, M. C., et al., Quantifying adenovirus-neutralizing antibodies by luciferase transgene detection: addressing preexisting immunity to vaccine and gene therapy vectors. J Clin Microbiol, 2003. 41(11): p. 5046-52.
19. Waddington, S. N., et al., Adenovirus serotype 5 hexon mediates liver gene transfer. Cell, 2008. 132(3): p. 397-409.
20. Liu, J., et al., Magnitude and phenotype of cellular immune responses elicited by recombinant adenovirus vectors and heterologous prime-boost regimens in rhesus monkeys. J Virol, 2008. 82(10): p. 4844-52.
21. Liu, J., et al., Immune control of an SIV challenge by a T-cell-based vaccine in rhesus monkeys. Nature, 2009. 457(7225): p. 87-91.
22. Lore, K., et al., Myeloid and plasmacytoid dendritic cells are susceptible to recombinant adenovirus vectors and stimulate polyfunctional memory T cell responses. J Immunol, 2007. 179(3): p. 1721-9.
23. unpublished, Barouch. et al.
24. Barouch et al., in AIDS Vaccine. 2009. Paris, France.
25. Kuschner, R. A., et al., A phase 3, randomized, double-blind, placebo-controlled study of the safety and efficacy of the live, oral adenovirus type 4 and type 7 vaccine, in U.S. military recruits. Vaccine, 2013. 31(28): p. 2963-71.
26. Masopust, D. and L. J. Picker, Hidden memories: frontline memory T cells and early pathogen interception. J Immunol, 2012. 188(12): p. 5811-7.
27. Bell, J. A., et al., Illness and microbial experiences of nursery children at junior village. American Journal of Hygiene, 1961. 74: p. 267-292.
28. Rhee, E. G. and D. H. Barouch, Adenoviruses, in Principles and Practice of Infectious Diseases, G. L. Mandell, J. E. Bennett, and R. Dolin, Editors. 2010, Elsevier: Philadelphia, Pa.
29. Brandt, C. D., et al., Infections in 18,000 infants and children in a controlled study of respiratory tract disease. I. Adenovirus pathogenicity in relation to serologic type and illness syndrome. Am J Epidemiol, 1969. 90(6): p. 484-500.
30. Fox, J. P., et al., The virus watch program: a continuing surveillance of viral infections in metropolitan New York families. VI. Observations of adenovirus infections: virus excretion patterns, antibody response, efficiency of surveillance, patterns of infections, and relation to illness. Am J Epidemiol, 1969. 89(1): p. 25-50.
31. Fox, J. P., C. E. Hall, and M. K. Cooney, The Seattle Virus Watch. VII. Observations of adenovirus infections. Am J Epidemiol, 1977. 105(4): p. 362-86.
32. Noel, J., et al., Identification of adenoviruses in faeces from patients with diarrhoea at the Hospitals for Sick Children, London, 1989-1992. J Med Virol, 1994. 43(1): p. 84-90.
33. Faden, H., et al., Pediatric adenovirus infection: relationship of clinical spectrum, seasonal distribution, and serotype. Clin Pediatr (Phila), 2011. 50(6): p. 483-7.
34. Abbas, K. Z., et al., Temporal changes in respiratory adenovirus serotypes circulating in the greater Toronto area, Ontario, during December 2008 to April 2010. Virol J, 2013. 10: p. 15.
35. Diarrhea: Why children are still dying and what can be done, 2009, The United Nations Children's Fund (UNICEF)/World Health Organization (WHO): New York, N.Y.
36. Ramani, S. and G. Kang, Viruses causing childhood diarrhoea in the developing world. Curr Opin Infect Dis, 2009. 22(5): p. 477-82.
37. Kotloff, K. L., et al., Burden and aetiology of diarrhoeal disease in infants and young children in developing countries (the Global Enteric Multicenter Study, GEMS): a prospective, case-control study. Lancet, 2013. 382(9888): p. 209-22.
38. Magwalivha, M., et al., High prevalence of species D human adenoviruses in fecal specimens from Urban Kenyan children with diarrhea. J Med Virol, 2010. 82(1): p. 77-84.
39. Liu, L. Y., et al., [Investigation of adenovirus infection in hospitalized children with diarrhea during 2010 in Beijing, China]. Zhonghua Er Ke Za Zhi, 2012. 50(6): p. 450-4.
40. Ouyang, Y., et al., Etiology and epidemiology of viral diarrhea in children under the age of five hospitalized in Tianjin, China. Arch Virol, 2012. 157(5): p. 881-7.
41. Lee, J. I., et al., Detection and molecular characterization of adenoviruses in Korean children hospitalized with acute gastroenteritis. Microbiol Immunol, 2012. 56(8): p. 523-8.
42. Espinola, E. E., et al., Genetic diversity of human adenovirus in hospitalized children with severe acute lower respiratory infections in Paraguay. J Clin Virol, 2012. 53(4): p. 367-9.
43. Mast, T. C., et al., International epidemiology of human pre-existing adenovirus (Ad) type-5, type-6, type-26 and type-36 neutralizing antibodies: correlates of high Ad5 titers and implications for potential HIV vaccine trials. Vaccine, 2010. 28: p. 950-957.
44. Kasel, J. A., et al., Conjunctivitis and enteric infection with adenovirus types 26 and 27: responses to primary, secondary and reciprocal cross-challenges. Am J Hyg, 1963. 77: p. 265-82.
45. Hierholzer, J. C., et al., Adenoviruses from patients with AIDS: a plethora of serotypes and a description of five new serotypes of subgenus D (types 43-47). J Infect Dis, 1988. 158(4): p. 804-13.
46. Khoo, S. H., et al., Adenovirus infections in human immunodeficiency virus-positive patients: clinical features and molecular epidemiology. J Infect Dis, 1995. 172(3): p. 629-37.
47. Curlin, M. E., et al., Frequent detection of human adenovirus from the lower gastrointestinal tract in men who have sex with men. PLoS One, 2010. 5(6): p. e11321.
48. Dubberke, E. R., et al., Acute meningoencephalitis caused by adenovirus serotype 26. J Neurovirol, 2006. 12(3): p. 235-40.
49. Koneru, B., et al., Adenoviral infections in pediatric liver transplant recipients. JAMA, 1987. 258(4): p. 489-92.
50. Venard, V., et al., Genotyping of adenoviruses isolated in an outbreak in a bone marrow transplant unit shows that diverse strains are involved. J Hosp Infect, 2000. 44(1): p. 71-4.
51. Al Qurashi, Y. M., M. Guiver, and R. J. Cooper, Sequence typing of adenovirus from samples from hematological stem cell transplant recipients. J Med Virol, 2011. 83(11): p. 1951-8.
52. Janes, H., et al., MRKAd5 HIV-1 Gag/Pol/Nef vaccine-induced T-cell responses inadequately predict distance of breakthrough HIV-1 sequences to the vaccine or viral load. PLoS One, 2012. 7(8): p. e43396.
53. Fischer, W., et al., Polyvalent vaccines for optimal coverage of potential T-cell epitopes in global HIV-1 variants. Nat Med, 2007. 13(1): p. 100-6.
54. Barouch, D. H., et al., Mosaic HIV-1 vaccines expand the breadth and depth of cellular immune responses in rhesus monkeys. Nat Med, 2010. 16(3): p. 319-23.
55. Santra, S., et al., Mosaic vaccines elicit CD8+T lymphocyte responses that confer enhanced immune coverage of diverse HIV strains in monkeys. Nat Med, 2010. 16(3): p. 324-8.
56. Li, Q., et al., Visualizing antigen-specific and infected cells in situ predicts outcomes in early viral infection. Science, 2009. 323(5922): p. 1726-9.
57. Baden, L. R., et al., First-in-human evaluation of the safety and immunogenicity of a recombinant adenovirus serotype 26 HIV-1 Env vaccine (IPCAVD 001). J Infect Dis, 2013. 207(2): p. 240-7.
58. Barouch, D. H., et al., Characterization of humoral and cellular immune responses elicited by a recombinant adenovirus serotype 26 HIV-1 Env vaccine in healthy adults (IPCAVD 001). J Infect Dis, 2013. 207(2): p. 248-56.
59. WO 2010/042942 entitled “Biochemically stabilized HIV-1 ENV Trimer Vaccine”
60. Kovacs et al, “HIV-1 envelope trimer elicits more potent neutralizing antibody responses than monomeric gp120,” PNAS 2012, 109(30):12111-6.
61. Havenga, et al., (2006) J Gen Virol 87 (Pt 8): 2135-43.
62. Jin, et al., Vaccine 28(27): 4369-75.
63. de Gruijl, et al., (2006) J Immunol 177(4): 2208-15.
64. Haslett et al. Journal of Infectious Diseases 181: 1264-72 (2000), page 1268.
65. Barouch et al., Cell 155:1-9, 2013.
66. Yang et al. (2002) J. Virol. 76:4634.
67. Chen et al. (2004) J. Virol. 78:4508.
68. Nkolola et al., 2010. Breadth of Neutralizing Antibodies Elicited by Stable, Homogeneous Clade A and Clade C HIV-1 gp140 Envelope Trimers in Guinea Pigs. J. Virology 84 (7): 3270-3279.
69. Cohen et al, 2002, J Gen Virol 83: 151-55.
70. Kobinger et al, 2006, Virology 346: 394-401.
71. Tatsis et al., 2007, Molecular Therapy 15: 608-17.
72. Bangari and Mittal, 2006, Vaccine 24: 849-62.
73. Lasaro and Ertl, 2009, Mol Ther 17: 1333-39.
74. Mayr et al. (1975), Infection 3, 6-14.
75. Mayr, A. & Danner, K. (1978), Dev. Biol. Stand. 41: 225-234.
76. Mayr et al. (1978), Zentralbl. Bacteriol. (B) 167:375-390.
77. Stickl (1974), Prev. Med. 3: 97-101.
78. Stickl and Hochstein-Mintzel (1971), Munch. Med. Wochenschr. 113: 1149-1153.
79. Blanchard et al. (1998), J. Gen. Virol. 79:1159-1167.
80. Carroll & Moss (1997), Virology 238:198-211.
81. Ambrosini et al. (1999), J. Neurosci. Res. 55: 569.
82. Boukamp et al (1988), J. Cell Biol. 106: 761-771.
83. J. Sambrook et al., Cold Spring Harbor Laboratory Press (1989).
84. Day and Kublin (2013), Curr HIV Res. 11(6): 441-449.

1. A method of inducing a safe and effective immune response against multiple clades of human immunodeficiency virus (HIV) in a human subject in need thereof, comprising:
(1) administering to the subject a priming composition comprising one or more Ad26 vectors together encoding at least three HIV antigenic polypeptides having the amino acid sequences of SEQ ID NO: 1, SEQ ID: NO 3, and SEQ ID NO: 4, and a pharmaceutically acceptable carrier, in a total dose of about 5×10⁹ to about 1×10¹¹ viral particles (vp) of the Ad26 vectors;
(2) administering to the subject, about 10-14 weeks after (1), the priming composition at a total dose of about 5×10⁹ to about 1×10¹¹ vp of the Ad26 vectors;
(3) administering to the subject, about 22-26 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 125 μg to 350 μg of the at least one isolated HIV envelope glycoprotein;
(4) administering to the subject, together with (3), a second boosting composition comprising one or more Ad26 vectors together encoding the at least three HIV antigenic polypeptides and a pharmaceutically acceptable carrier, at a total dose of about 5×10⁹ to about 1×10¹¹ vp of the Ad26 vectors; or
administering to the subject, together with (3), a second alternative boosting composition comprising one or more MVA vectors together encoding the at least three HIV antigenic polypeptides, and a pharmaceutically acceptable carrier, at a total dose of about 10⁷ to about 10⁹ plaque-forming units (pfu) of the MVA vectors,
wherein the safe and effective immune response comprises an antibody-dependent cellular phagocytosis (ADCP) response to isolated HIV envelope glycoproteins from clades B and C at a median response rate of at least 56%.

2. The method of claim 1, further comprising:
(5) administering to the subject, about 42-60 weeks after (1), the first boosting composition at a total dose of about 125 μg to 350 μg, of the at least one isolated HIV envelope glycoprotein; and
(6) administering to the subject, together with (5), the second boosting composition at a total dose of about 5×10⁹ to about 1×10¹¹ vp of the Ad26 vectors; or
administering to the subject, together with (5), the second alternative boosting composition at a total dose of about 10⁷ to about 10⁹ pfu of the MVA vectors.

3. The method of claim 1, wherein the priming composition and the second boosting composition each comprise at least three rAd26 vectors encoding the at least three HIV antigenic polypeptides.

4. The method of claim 1, wherein at step (1) the human subject is uninfected by HIV.

5. The method of claim 1, wherein the safe and effective immune response further comprises an ADCP response to isolated HIV envelope glycoprotein from clade A at a median response rate of at least 35%.

6. The method of claim 1, comprising:
(1) administering to the subject a priming composition comprising at least three Ad26 vectors together encoding at least three HIV antigenic polypeptides having the amino acid sequences of SEQ ID NO: 1, SEQ ID: NO 3, and SEQ ID NO: 4, respectively, and a pharmaceutically acceptable carrier, in a total dose of about 5×10¹⁰ viral particles (vp) of the Ad26 vectors;
(2) administering to the subject, about 12 weeks after (1), the priming composition at a total dose of about 5×10¹⁰ vp of the Ad26 vectors;
(3) administering to the subject, about 24 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 250 μg of the at least one isolated HIV envelope glycoprotein;
(4) administering to the subject, together with (3), a second boosting composition comprising the at least three Ad26 vectors and a pharmaceutically acceptable carrier, at a total dose of about 5×10¹⁰ vp of the Ad26 vectors;
(5) administering to the subject, about 48 weeks after (1), the first boosting composition at a total dose of about 250 μg of the at least one isolated HIV envelope glycoprotein; and
(6) administering to the subject, together with (5), the second boosting composition at a total dose of about 5×10¹⁰ vp of the Ad26 vectors;
wherein the safe and effective immune response comprises an antibody-dependent cellular phagocytosis (ADCP) response to isolated HIV envelope glycoproteins of clades B and C at a median response rate of at least 56%.

7. The method of claim 6, wherein the ADCP response against HIV envelope glycoproteins of clade B and C is at a median response rate of at least about 70%.

8. The method of claim 6, wherein the safe and effective immune response further comprises a humoral immune response against HIV envelope glycoprotein from clades A, B, and C at a median response rate of at least 90% as measured by IgG bindings to HIV envelope glycoproteins from clades A, B and C in enzyme-linked immunosorbent assays (ELISAs).

9. The method of claim 6, wherein the safe and effective immune response further comprises a cellular immune response at a median response rate of at least 50% as measured by a γIFN response in an enzyme-linked immunospot assay (ELISPOT) to a potential T-cell epitopes (PTE) peptide pool.

10. The method of claim 9, wherein the cellular immune response has a median response rate of at least about 70%.

11. A method of inducing a safe and effective immune response against multiple clades of human immunodeficiency virus (HIV) in a human subject in need thereof, comprising:
(1) administering to the subject a priming composition comprising one or more Ad26 vectors together encoding at least three HIV antigenic polypeptides having the amino acid sequences of SEQ ID NO: 1, SEQ ID: NO 3, and SEQ ID NO: 4, and a pharmaceutically acceptable carrier, in a total dose of about 5×10⁹ to about 1×10¹¹ viral particles (vp);
(2) administering to the subject, about 10-14 weeks after (1), the priming composition at a total dose of about 5×10⁹ to about 1×10¹¹ vp of the Ad26 vectors;
(3) administering to the subject, about 22-26 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 125 μg to 350 μg of the at least one isolated HIV envelope glycoprotein;
(4) administering to the subject, together with (3), a second boosting composition comprising one or more Ad26 vectors encoding the at least three HIV antigenic polypeptides and a pharmaceutically acceptable carrier, at a total dose of about 5×10⁹ to about 1×10¹¹ vp of the Ad26 vectors; or
administering to the subject, together with (3), a second alternative boosting composition comprising one or more MVA vectors encoding the at least three HIV antigenic polypeptides, and a pharmaceutically acceptable carrier, at a total dose of about 10⁷ to about 10⁹ plaque-forming units (pfu) of the MVA vectors,
wherein the safe and effective immune response comprises a humoral immune response against HIV clades A, B and C at a median response rate of at least 90%, as measured by IgG bindings to HIV envelope glycoproteins from clades A, B and C in enzyme-linked immunosorbent assays (ELISAs).

12. The method of claim 11, comprising:
(1) administering to the subject a priming composition comprising at least three Ad26 vectors together encoding at least three HIV antigenic polypeptides having the amino acid sequences of SEQ ID NO: 1, SEQ ID: NO 3, and SEQ ID NO: 4, respectively, and a pharmaceutically acceptable carrier, in a total dose of about 5×10¹⁰ viral particles (vp) of the Ad26 vectors;
(2) administering to the subject, about 12 weeks after (1), the priming composition at a total dose of about 5×10¹⁰ vp of the Ad26 vectors;
(3) administering to the subject, about 24 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 250 μg of the at least one isolated HIV envelope glycoprotein;
(4) administering to the subject, together with (3), a second boosting composition comprising the at least three Ad26 vectors and a pharmaceutically acceptable carrier, at a total dose of about 5×10¹⁰ vp of the Ad26 vectors;
(5) administering to the subject, about 48 weeks after (1), the first boosting composition at a total dose of about 250 μg of the at least one isolated HIV envelope glycoprotein; and
(6) administering to the subject, together with (5), the second boosting composition at a total dose of about 5×10¹⁰ vp of the Ad26 vectors;
wherein the safe and effective immune response comprises a humoral immune response against HIV clades A, B and C at a median response rate of at least 90%, as measured by IgG bindings to HIV envelope glycoproteins from clades A, B and C in enzyme-linked immunosorbent assays (ELISAs).

13. A method of inducing a safe and effective immune response against multiple clades of human immunodeficiency virus (HIV) in a human subject in need thereof, comprising:
(1) administering to the subject a priming composition comprising one or more Ad26 vectors together encoding at least three HIV antigenic polypeptides having the amino acid sequences of SEQ ID NO: 1, SEQ ID: NO 3, and SEQ ID NO: 4, and a pharmaceutically acceptable carrier, in a total dose of about 5×10⁹ to about 1×10¹¹ viral particles (vp) of the Ad26 vectors;
(2) administering to the subject, about 10-14 weeks after (1), the priming composition at a total dose of about 5×10⁹ to about 1×10¹¹ vp of the Ad26 vectors;
(3) administering to the subject, about 22-26 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 150 μg to 350 μg of the isolated HIV envelope glycoprotein;
(4) administering to the subject, together with (3), a second boosting composition comprising one or more Ad26 vectors encoding the at least three HIV antigenic polypeptides and a pharmaceutically acceptable carrier, at a total dose of about 5×10⁹ to about 1×10¹¹ vp of the Ad26 vectors; or
administering to the subject, together with (3), a second alternative boosting composition comprising one or more MVA vectors encoding the at least three HIV antigenic polypeptides, and a pharmaceutically acceptable carrier, at a total dose of about 10⁷ to about 10⁹ plaque-forming units (pfu) of the MVA vectors,
wherein the safe and effective immune response comprises a cellular immune response at a median response rate of at least 50%, as measured by a gammaIFN response in an ELISPOT to a potential T-cell epitopes (PTE) peptide pool.

14. The method of claim 13, comprising:
(1) administering to the subject a priming composition comprising at least three Ad26 vectors together encoding at least three HIV antigenic polypeptides having the amino acid sequences of SEQ ID NO: 1, SEQ ID: NO 3, and SEQ ID NO: 4, respectively, and a pharmaceutically acceptable carrier, in a total dose of about 5×10¹⁰ viral particles (vp) of the Ad26 vectors;
(2) administering to the subject, about 12 weeks after (1), the priming composition at a total dose of about 5×10¹⁰ vp of the Ad26 vectors;
(3) administering to the subject, about 24 weeks after (1), a first boosting composition comprising at least one isolated HIV envelope glycoprotein having the amino acid sequence of SEQ ID NO: 5, an aluminum phosphate adjuvant and a pharmaceutically acceptable carrier, at a total dose of about 250 μg of the at least one isolated HIV envelope glycoprotein;
(4) administering to the subject, together with (3), a second boosting composition comprising the at least three Ad26 vectors and a pharmaceutically acceptable carrier, at a total dose of about 5×10¹⁰ vp of the Ad26 vectors;
(5) administering to the subject, about 48 weeks after (1), the first boosting composition at a total dose of about 250 μg of the at least one isolated HIV envelope glycoprotein; and
(6) administering to the subject, together with (5), the second boosting composition at a total dose of about 5×10¹⁰ vp of the Ad26 vectors;
wherein the safe and effective immune response comprises a cellular immune response at a median response rate of at least 50%, as measured by a gammaIFN response in an ELISPOT to a potential T-cell epitopes (PTE) peptide pool.

15. The method according to claim 1, wherein the safe and effective immune response comprises a persistent humoral immune response against HIV envelope glycoprotein from at least Clade C at a response rate of at least 90% at 48 weeks after the last administration of the boosting compositions.

16. The method according to claim 6, wherein the safe and effective immune response comprises a persistent humoral immune response against HIV envelope glycoprotein from at least Clade C at a response rate of at least 90% at 48 weeks after the last administration of the boosting compositions.

17. The method according to claim 11, wherein the safe and effective immune response comprises a persistent humoral immune response against HIV envelope glycoprotein from at least Clade C at a response rate of at least 90% at 48 weeks after the last administration of the boosting compositions.

18. The method according to claim 12, wherein the safe and effective immune response comprises a persistent humoral immune response against HIV envelope glycoprotein from at least Clade C at a response rate of at least 90% at 48 weeks after the last administration of the boosting compositions.

19. The method according to claim 13, wherein the safe and effective immune response comprises a persistent humoral immune response against HIV envelope glycoprotein from at least Clade C at a response rate of at least 90% at 48 weeks after the last administration of the boosting compositions.

20. The method according to claim 14, wherein the safe and effective immune response comprises a persistent humoral immune response against HIV envelope glycoprotein from at least Clade C at a response rate of at least 90% at 48 weeks after the last administration of the boosting compositions.


SEQUENCE LISTING (8 sequences)

SEQ ID NO: 1 - protein, 685 aa, Artificial Sequence
  Mos1.Env mosaic antigen sequence
MRVTGIRKNYQHLWRWGTMLLGILMICSAAGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKRRVVQSEKSAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLW

SEQ ID NO: 2 - protein, 684 aa, Artificial Sequence
  Mos2.Env mosaic antigen sequence
MRVRGIQRNWPQWWIWGILGFWMIIICRVMGNLWVTVYYGVPVWKEAKTTLFCASDAKAYEKEVHNVWATHACVPTDPNPQEMVLENVTENFNMWKNDMVDQMHEDIIRLWDQSLKPCVKLTPLCVTLECRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTINFTSSSGGDLEITTHSFNCRGEFFYCNTSGLFNGTYMPNGTNSNSSSNITLPCRIKQIINMWQEVGRAMYAPPIAGNITCRSNITGLLLTRDGGSNNGVPNDTETFRPGGGDMRNNWRSELYKYKVVEVKPLGVAPTEAKRRVVESEKSAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWKNLWNWFDITNWLW

SEQ ID NO: 3 - protein, 1350 aa, Artificial Sequence
  Mos1.Gag-Pol mosaic antigen sequence
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 4 - protein, 1341 aa, Artificial Sequence
  Mos2.Gag-Pol mosaic antigen sequence
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQMAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 5 - protein, 679 aa, Artificial Sequence
  clade C gp140 trimeric protein
AENLWVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGAKERVVEREERAVGIGAVFLGFLGAAGSTMGAASLTLTVQARQLLSSIVQQQSNLLRAIEAQQHMLQLTVWGIKQLQTRVLAIERYLKDQQLLGIWGCSGKLICTTNVPWNSSWSNKSQTDIWNNMTWMEWDREISNYTDTIYRLLEDSQTQQEKNEKDLLALDSWKNLWSWFDISNWLWYIKSRIEGRGSGGYIPEAPRDGQAYVRKDGEWVLLSTFL

SEQ ID NO: 6 - protein, 695 aa, Artificial Sequence
  mosaic gp140 chimeric protein
AGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKERVVQREERAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLWYIKSRIEGRGSGGYIPEAPRDGQAYVRKDGEWVLLSTFL

SEQ ID NO: 7 - protein, 29 aa, Artificial Sequence
  exemplary leader sequence for gp140 stabilized trimeric protein production
MRVRGIQRNCQHLWRWGTLILGMLMICSA

SEQ ID NO: 8 - protein, 711 aa, Artificial Sequence
  mos2S Env antigen sequence
MRVRGMLRNWQQWWIWSSLGFWMLMIYSVMGNLWVTVYYGVPVWKDAKTTLFCASDAKAYEKEVHNVWATHACVPTDPNPQEIVLGNVTENFNMWKNDMVDQMHEDIISLWDASLEPCVKLTPLCVTLNCRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTIKFAPHSGGDLEITTHTFNCRGEFFYCNTSNLFNESNIERNDSIITLPCRIKQIINMWQEVGRAIYAPPIAGNITCRSNITGLLLTRDGGSNNGVPNDTETFRPGGGDMRNNWRSELYKYKVVEVKPLGVAPTEAKRRVVEREKRAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWNNLWNWFSISKWLWYIKIFIMIVGGLIGLRIIFAVLSIVNRVRQGY